(19) United States
(12) Patent Application Publication
(10) Pub. No.: US 2025/0170350 A1
(43) Pub. Date: May 29, 2025

(54) POSITIVE AIRWAY PRESSURE DEVICE

(71) Applicant: Leslie C. HOFFMANN, Tarzana, CA (US)

(72) Inventor: Leslie C. HOFFMANN, Tarzana, CA (US)

(21) Appl. No.: 18/692,288

(22) PCT Filed: Sep. 14, 2022
(86) PCT No.: PCT/US2022/043556

§ 371 Date: Mar. 14, 2024

Related U.S. Application Data
(60) Provisional application No. 63/261,200, filed on Sep. 14, 2021.
(60) Provisional application No. 63/316,351, filed on Mar. 3, 2022.

(51) Int. Cl. A61M 16/06; A61M 16/00

(52) U.S. Cl. CPC A61M 16/0605; A61M 16/0066; A61M 2205/42; A61M 2205/8206

(57) ABSTRACT

A body-worn PAP system providing proper mask fitment during body movement, reduced leakage, and elimination of a need for a connecting hose to the blower unit, and a PAP blower adapted for use in a PAP system with a compact noise reduction system that does not utilize acoustic foams in the airflow path.

The present application claims the benefit of U.S. Provisional Application 63/261,200, filed Sep. 14, 2021, and U.S. Provisional Application 63/316,351, filed Mar. 3, 2022, each of which is hereby incorporated in its entirety and from each of which priority is claimed.

### FIELD OF THE INVENTION

The present invention relates to the methods and devices for the treatment of airway disorders using positive airway pressure.

### BACKGROUND OF THE INVENTION

The following discussion of the background of the invention is merely provided to aid the reader in understanding the invention and is not admitted to describe or constitute prior art to the present invention.

Positive airway pressure (PAP) therapy is a generic term applied to treatments using a stream of air at greater than atmospheric pressure to support airway and pulmonary patency. Typical PAP therapy involves a portable machine that directs pressurized room air into the airway through a tube connected to a face mask. This positive airflow helps keep the airway open. The most familiar application of PAP therapy is preventing the collapse that occurs during sleep apnea, thus allowing normal breathing.

There are various types of PAP therapy that may be employed. Continuous positive airway pressure (CPAP) is a type of PAP therapy in which the air flow is introduced into the airways to maintain a continuous pressure to constantly stent the airways open, in people who are breathing spontaneously. In bilevel positive airway pressure (BiPAP), the air pressure cycles between two levels of pressure based on whether the patient is inhaling or exhaling, but positive pressure is delivered continuously at some level. These pressures are known as inspiratory positive airway pressure (IPAP) and expiratory positive airway pressure (EPAP).

The pressures involved are typically recited in terms of centimeters of water pressure (cm H₂O) because lung pressure is often expressed in those units. 1 cm H₂O is the amount of pressure required to raise a column of water 1 cm in height. This is equal to 98.07 Pascals. The pressures utilized in PAP therapy are generally between 2 and 30 cm H₂O. Various interfaces are available for the delivery of PAP and most can be divided into 3 categories: nasal masks which cover the nose and leave the mouth free; nasal pillows which insert directly into the nostrils; and oronasal (i.e., full face) masks, which cover the nose and the mouth. These interfaces are collectively referred to herein as “face masks.”

Despite the high efficacy of PAP to reverse upper airway obstruction in sleep apnea, treatment effectiveness is limited by variable adherence to prescribed therapy. When adherence is defined as greater than 4 hours of nightly use, 46 to 83% of patients with obstructive sleep apnea have been reported to be nonadherent to treatment. Evidence suggests that use of PAP for longer than 6 hours decreases sleepiness, improves daily functioning, and restores memory to normal levels. The decision to embrace PAP occurs during the first few days of treatment. Typical reasons cited for non-compliance are claustrophobia from the face mask, inability to tolerate compressed air flow and pressure, noise, poor face mask fit/leaks, difficulty sleeping with the device and its long mask connecting hose, and co-sleeping partner disruption. No single factor has been consistently identified as predictive of adherence.

### BRIEF DESCRIPTION OF THE INVENTION

It is an object of the invention to provide a body-worn PAP system providing proper mask fitment during body movement, reduced leakage, and elimination of a need for a connecting hose to the blower unit. It is a second object of the invention to provide a PAP blower adapted for use in a PAP system with a compact noise reduction system that does not need acoustic foams in the airflow path.

In a first aspect, the invention relates to a flow generator adapted for use in a PAP system. The flow generator comprises:
a housing;
an air inlet aperture formed in a wall of the housing configured to allow atmospheric air into the housing;
an air path within the housing in fluid communication with the air inlet aperture, comprising
   a first chamber proximal to the air inlet aperture having a substantially rectangular cuboid shape,
   a first flow straightener comprising a first array of substantially parallel air passages downstream of, and in fluid communication with, the first chamber,
   a second chamber immediately downstream of, and in fluid communication with, the first air straightener having a substantially rectangular cuboid shape, and
   a second flow straightener comprising a second array of parallel air passages downstream of, and in fluid communication with, the second chamber,
   a third chamber immediately downstream of, and in fluid communication with, the second air straightener, and
   a fourth chamber within the housing downstream of, and in fluid communication with, the third chamber;
a blower within the fourth chamber comprising an impeller and a motor; and
an air outlet aperture formed in a wall of the housing operably connected to the blower to receive air exiting an air output portion of the blower,
wherein the blower, when energized, is configured to draw atmospheric air into the inlet aperture and thereby generate an airflow through the air path and into the blower, and direct the airflow at greater than atmospheric pressure to the air outlet aperture.

As is apparent from the foregoing, according to the present invention, the air path comprises a plurality of flow straighteners, each followed by an associated chamber volume, between the first chamber proximal to the air inlet aperture and the chamber comprising a blower. Each chamber volume following a flow straightener is an unoccupied space in the air path. In certain embodiments, the flow straighteners occupy between 50% and 99% of the volume of the air path between the air inlet aperture and the fourth (blower) chamber, without accounting for the volume contained in the array of parallel air passages (meaning, considering the flow straighteners as if the parallel air passages did not exist and the flow straighteners were simply solid blocks). There may be 1, 2, 3, 4, 5 or more flow straighteners arranged in a serial fashion, each interleaved by an associated chamber. The volume of the chamber between flow straighteners is determined by the spacing between flow straighteners. This spacing may be as small as 0.1 mm to several centimeters. Each air straightener is preferably at least 18 mm in length as measured along the air passages.

In certain embodiments, the flow generator comprises one or more additional flow straighteners in the air path prior to the fourth chamber. By way of example only, then, the flow generator comprises:
a housing;
an air inlet aperture formed in a wall of the housing configured to allow atmospheric air into the housing;
an air path within the housing in fluid communication with the air inlet aperture, comprising
   a first chamber proximal to the air inlet aperture having a substantially rectangular cuboid shape,
   a first flow straightener comprising a first array of substantially parallel air passages downstream of, and in fluid communication with, the first chamber,
   a second chamber immediately downstream of, and in fluid communication with, the first air straightener having a substantially rectangular cuboid shape, and
   a second flow straightener comprising a second array of parallel air passages downstream of, and in fluid communication with, the second chamber,
   a third chamber immediately downstream of, and in fluid communication with, the second air straightener,
   one or more additional flow straighteners comprising an array of parallel air passages downstream of, and in fluid communication with, the second chamber, wherein each of the one or more additional flow starighteners is followed by a chamber immediately downstream thereof, and
   a fourth chamber within the housing immediately downstream of, and in fluid communication with, the chamber following the additional flow straighteners;
a blower within the fourth chamber comprising an impeller and a motor; and
an air outlet aperture formed in a wall of the housing operably connected to the blower to receive air exiting an air output portion of the blower,
wherein the blower, when energized, is configured to draw atmospheric air into the inlet aperture and thereby generate an airflow through the air path and into the blower, and direct the airflow at greater than atmospheric pressure to the air outlet aperture.

In certain embodiments, there are one, two, three, or more of the additional flow straighteners in the air path. In preferred embodiments, the flow generator comprises:
a housing;
an air inlet aperture formed in a wall of the housing configured to allow atmospheric air into the housing;
an air path within the housing in fluid communication with the air inlet aperture, comprising
   a first chamber proximal to the air inlet aperture having a substantially rectangular cuboid shape,
   a first, second, and third flow straightener comprising a first array of substantially parallel air passages, wherein the first flow straightener is downstream of, and in fluid communication with, the first chamber, the second flow straightener comprising a second array of parallel air passages is downstream of, and in fluid communication with the first flow straightener, and the third flow straightener comprising a third array of parallel air passages is downstream of, and in fluid communication with the second flow straightener,
   a chamber following each flow chamber, wherein the chamber is an unoccupied space in the air path, and
   a blower chamber within the housing immediately downstream of, and in fluid communication with, the chamber following the third flow straightener;
   a blower within the blower chamber comprising an impeller and a motor; and
   an air outlet aperture formed in a wall of the housing operably connected to the blower to receive air exiting an air output portion of the blower,
wherein the blower, when energized, is configured to draw atmospheric air into the inlet aperture and thereby generate an airflow through the air path and into the blower, and direct the airflow at greater than atmospheric pressure to the air outlet aperture, wherein the spacing between the first and second flow straighteners and the spacing between the second and third flow straighteners are each independently between 0.1 and 0.3 mm, and wherein the first, second, and third flow straighteners together occupy between 75% and 99% of the volume between the air inlet aperture and the blower chamber, measured without accounting for the volume occupied by the array of parallel air passages in each flow straightener.

The air passages in the flow straighteners are preferably linear or substantially linear. By “substantially linear” is meant an air passage which deviates from linear by no more than 20% and may curve in a smooth manner that lacks an edge or a sudden angular change of direction.

In certain embodiments, each of the air passages in the flow straighteners are substantially identical in cross-section. The shape may be any shape, although a substantially hexagonal cross-section is preferred. In certain embodiments, one or more, and in certain embodiments all, air passage in a flow straightener is substantially “in line” with a corresponding air passage in the ajoining flow straightener, meaning that a single linear axis can traverse the two corresponding flow straightener. Regular arrays of 6 or 7 hexagonal holes with an approximate 3 mm side to side dimension across the flats, arranged as shown in the figures are preferred examples.

In certain embodiments, the plurality of flow straighteners alternate in hole number. By way of example, if the first flow straightener may comprise 7 air passages, the second flow straightener would comprise 6 air passages, and the third flow straightener would comprise 7 air passages, resulting in a 7-6-7 alternating pattern of (preferably hexagonal) air passages in the three flow straighteners. In these embodiments, one or more of the air passages are substantially “in line” with corresponding air passages in the ajoining flow straightener, while at least one air passage in the “7” flow straightener is not in line with a corresponding air passage in the ajoining “6” flow straightener

In various embodiments, a flow generator of the present invention has at least one, and preferably 2, 3, 4, 5, or all, of the following characteristics:
i) the volume of the first chamber can be minimal or up to 6,500 mm³ as space allows,
ii) the volume of the second chamber can be minimal (e.g., 0.1 mm) or up to 10,000 mm³,
iii) the volume of the third chamber ahead of an optional third air straightener can be minimal (e.g., 0.1 mm) or up to 10,000 mm³,
iv) the volume of the final chamber connecting to the chamber containing the blower is approximately 4,000 to 14,000 mm³,
v) the length of each array of air passages in the air straightener elements is approximately 19 to 25 mm, and
vi) the cross-sectional area of each hexagonal flow passage in the first, second and optional third array of air passages is approximately 8 mm².

These dimensions may be scaled as appropriate, for example based on the size and/or air output of the blower selected. Preferably, these dimensions are not substantially altered in a manner that would substantially increase flow resistance through the device, for example by reducing the cross sectional area of the flow channels.

In certain embodiments, the central axis of the air path from the chamber volume after all the air straightener elements diverges from the central axis of the air path from the first flow chamber to the third flow chamber, meaning that the air flow follows a path into the chamber containing the blower element does not lie on the same single linear axis as the path traversed through the flow straighteners. By way of example, air which reaches the last flow chamber after the air straightener elements may be required to diverge at approximately 90° to the central axis of the air path through the flow straighteners in order to enter the chamber containing the blower element.

As described herein, the airflow through the flow generator preferably does not pass acoustic foam materials between the air inlet aperture and the air output aperture, as such foam may degrade and enter the air flow, thereby being inhaled or ingested by the wearer.

In certain embodiments, the chamber containing the blower element can comprise a resilient liner seated within the chamber, wherein the blower is adapted to seat in a soft and flexible raised retaining feature integrally or unitarily formed on a surface of the resilient liner. This resilient liner acts to both retain and cushion the blower from damage due to handling or dropping of the flow generator during manufacture, shipping, storage, or use. In preferred embodiments, the resilient liner is configured to conform to the shape of the chamber such that it is retained within the chamber without any adhesive. Such embodiments simplify manufacture of the flow generator.

In a second aspect, the invention provides a PAP system adapted for treatment of an individual having respiratory disease, sleep disordered breathing, or requiring breathing support, comprising:
a flow generator, comprising
   a housing,
   a blower comprising an impeller and a motor within the housing, and
   a flow outlet within the housing, wherein the flow generator is adapted to provide a flow of pressurized breathable gas produced by the blower through the flow outlet;
a power supply operably connected to the flow generator and adapted to provide electrical energy to energize the blower;
a securement interface, comprising
   a bracket projecting from the flow generator or the power supply, wherein the bracket is adapted to support and orient the flow generator or the power supply on the mental protuberance of the individual's mandible and to isolate the individual's cranial bones from noise and vibration generated during operation of the flow generator,
   at least two slots or other connection/securement means on the bracket configured and arranged to be on opposite sides of the individual's mental protuberance,
   one or more flexible head straps adapted to couple to the at least two slots on the bracket and to engage the head of the individual to secure the bracket on the individual's mental protuberance; and
a mask interface, comprising
   an interface portion in fluid communication with the flow outlet of the flow generator, and
   a face mask, where the face mask is preferably directly connected to the flow generator housing.

In certain embodiments, the PAP system comprises the flow generator as described in the first aspect.

The PAP system described herein may utilize any face mask known in the art as part of the mask interface. In certain embodiments, the face mask comprises a pair of nasal pillows adapted for at least partial insertion into the nares of the individual. In certain embodiments, the face mask is as depicted in FIGS. 10 and 11, which is described in detail hereinafter.

In embodiments where the bracket projects from the flow generator, the bracket preferably comprises a chin cup adapted to conform approximately to the shape of the mental protuberance. In certain embodiments, a chin cup insert which is adapted to form a cushioned contact surface between the chin cup and the mental protuberance forms a patient contact surface on the chin cup. A battery power supply may be integrated within the flow generator housing, however this power supply may be a separate component from the flow generator and connected by a cable providing an electrical connection between the power supply and the flow generator, or may also be located in close proximity to the flow generator housing and connected via a short lead. When the power supply is provided as a separate component, it may be in the form of a battery pack or as a connection to a mains power source (e.g., a plug-in transformer that receives 120 v mains power and provides DC voltage to the flow generator.

In embodiments where the bracket projects from the power supply, the power supply may be attached to an external surface of the flow generator housing such that the mass of the power supply is supported on and as close as possible to the mental protuberance. In certain embodiments, the flow generator is permitted to “float” relative to the power supply, thereby reducing movement of the flow generator (and so the patient interface) during movement of the wearer by reducing the torque generated by the relatively heavy batteries within the power supply.

In certain embodiments, the PAP system of the invention is configured to be a completely body-worn system. Preferably, the power supply comprises a battery as the source of electrical power to the flow generator. Most preferably, the energy usage of the PAP system can provide a continuous on-demand 10 cm H₂O pressure for at least 10 hours through a CPAP face mask. For comfortable use during sleep, this completely body-worn system preferably weighs less than 0.5 kg in use.

In one aspect, then, the invention provides a PAP system adapted for treatment of an individual having respiratory disease, sleep disordered breathing, or requiring breathing support, comprising:
a flow generator, comprising
   a housing,
   a blower comprising an impeller and a motor within the housing, and
   a flow outlet within the housing, wherein the flow generator is adapted to provide a flow of pressurized breathable gas produced by the blower through the flow outlet;
a power supply integrated within or in close proximity to the flow generator housing and operably connected to the flow generator and adapted to provide electrical energy to energize the blower;
a securement interface, comprising
   a bracket projecting from the flow generator, wherein the bracket is adapted to support and orient the flow generator on the mental protuberance of the individual's mandible and to isolate the individual's cranial bones from noise and vibration generated during operation of the flow generator,
   at least two slots or other connection/securement means on the bracket configured and arranged to be on opposite sides of the individual's mental protuberance,
   one or more flexible head straps adapted to couple to the at least two slots on the bracket and to engage the head of the individual to secure the bracket on the individual's mental protuberance;
a mask interface, comprising
   an interface portion in fluid communication with the flow outlet of the flow generator, and
   a face mask where the face mask is preferably directly connected to the flow generator housing.

In another aspect the invention provides a PAP system adapted for treatment of an individual having respiratory disease, sleep disordered breathing, or requiring breathing support, comprising:
a flow generator, comprising
   a housing,
   a blower comprising an impeller and a motor within the housing, and
   a flow outlet within the housing, wherein the flow generator is adapted to provide a flow of pressurized breathable gas produced by the blower through the flow outlet;
a power supply operably connected to the flow generator and adapted to provide electrical energy to energize the blower, wherein the power supply is separate from the flow generator and connected by a cable providing an electrical connection between the power supply and the flow generator; and
a securement interface, comprising
   a bracket projecting from the flow generator, wherein the bracket is adapted to support and orient the flow generator on the mental protuberance of the individual's mandible and to isolate the individual's cranial bones from noise and vibration generated during operation of the flow generator,
   at least two slots or other connection/securement means on the bracket configured and arranged to be on opposite sides of the individual's mental protuberance,
   one or more flexible head straps adapted to couple to the at least two slots on the bracket and to engage the head of the individual to secure the bracket on the individual's mental protuberance; and
a mask interface, comprising
   an interface portion in fluid communication with the flow outlet of the flow generator, and
   a face mask where the face mask is preferably directly connected to the flow generator housing.

In another aspect, the invention provides a PAP system adapted for treatment of an individual having respiratory disease, sleep disordered breathing, or requiring breathing support, comprising:
a flow generator, comprising
   a housing,
   at least two slots or other connection securement means on the housing;
   a blower comprising an impeller and a motor within the housing, and
   a flow outlet within the housing, wherein the flow generator is adapted to provide a flow of pressurized breathable gas produced by the blower through the flow outlet;
a power supply comprising a second housing and one or more batteries within the housing, wherein the power supply is operably connected to the flow generator and adapted to provide electrical energy from the one or more batteries to energize the blower and whereby the battery pack is removable to reduce weight if desired and the flow generator is powered by a flexible cord attached to an external power supply;
a securement interface, comprising
   a bracket projecting from the power supply, wherein the bracket is adapted to support and orient the power supply on the mental protuberance of the individual's mandible and to isolate the individual's cranial bones from noise and vibration generated during operation of the flow generator,
   at least two slots or other connection/securement means on the bracket configured and arranged to be on opposite sides of the individual's mental protuberance,
   one or more flexible head straps adapted to couple to the at least two slots on the bracket, to engage the head of the individual to secure the device on the individual's mental protuberance; and
a mask interface, comprising
   an interface portion in fluid communication with the flow outlet of the flow generator, and
   a face mask where the face mask is preferably directly connected to the flow generator housing.

In certain embodiments, the PAP system comprises the flow generator as described in the first aspect.

Most preferably, the energy usage of the PAP system can provide a continuous on-demand 10 cm H₂O pressure through a CPAP face mask. For comfortable use during sleep, this completely body-worn system weighs less than 0.3 kg in use without an attached battery.

In a related aspect, the present invention relates to methods of treating sleep apnea, snoring, or another breathing disorder in an individual, comprising:
securing a PAP system of the invention to the individual by positioning the bracket projecting from the flow generator onto the mental protuberance of the individual's mandible, engaging the head straps to the head of the individual to maintain the flow generator on the mental protuberance, and positioning the face mask over an external airway passage of the individual; and
energizing the flow generator to administer airflow produced by the flow generator into the individual's airway via the face mask.

### BRIEF DESCRIPTION OF THE FIGURES

FIG. 1 depicts an exploded drawing of a PAP system of the present invention.

FIG. 2 depicts various elevation views of the exemplary PAP system of the invention shown in FIG. 1.

FIG. 3 depicts various cross-section views through the exemplary PAP system of the invention shown in FIG. 1.

FIG. 4 depicts an exploded drawing of another PAP system of the present invention.

FIG. 5 depicts an exemplary flow straightener for use in a PAP system of the present invention.

FIG. 6 depicts the interior of the lower enclosure of a PAP system of the present invention with upper enclosure removed so that the blower can be shown.

FIG. 7 depicts an exemplary PAP system of the invention as worn on a user.

FIG. 8 depicts a resilient cushioning liner and blower support mounting of a PAP system of the present invention with the blower removed.

FIG. 9 depicts the back side (i.e., facing away from the wearer) of a chin cup for use on a PAP system of the present invention.

FIG. 10 depicts a PAP system of the present invention from the aspect facing the wearer.

FIG. 11 depicts one view of a PAP system patient interface, or “face mask.”

FIG. 12 depicts two exemplary PAP systems of the present invention.

FIG. 13 depicts detailed views of a PAP system patient interface of the present invention.

FIGS. 14 A and B depicts two views of another exemplary PAP systems of the present invention.

FIG. 15 depicts an exploded drawing of another exemplary PAP system of the present invention.

FIGS. 16 A and B depicts a sliding interface mount between the bracket (e.g., chin cup) and the power supply (e.g., a battery pack) in an unmounted (A) and mounted (B) state.

FIGS. 17 A and B depict two views of the interior of the lower enclosure of a PAP system of the present invention with upper enclosure removed so that the blower can be shown.

FIG. 18 depicts an exemplary air inlet cover.

FIG. 19 depicts another exemplary PAP system of the invention as worn on a user.

FIG. 20 depicts yet another exemplary PAP system of the invention as worn on a user.

FIG. 21 depicts an exploded drawing of another exemplary PAP system of the present invention having three flow straighteners.

### DETAILED DESCRIPTION OF THE INVENTION

The present invention provides wearable PAP systems for providing positive air pressure, particularly for use in Continuous Positive Airway Pressure (CPAP) devices, bilevel pressure support devices (BiPAP) devices and related respiratory support devices. As described hereinafter, the present systems provide simple and inexpensive assembly with mass-producible parts. The design effectively isolates offensive noise and vibration from being transmitted to the wearer of the device without the use of acoustic abatement foams that can contaminate the air pathway and be subsequently inhaled or ingested by the user. Including any batteries required for operation, the systems of the invention preferably weigh less than 0.5 kg and the device housing, excluding the nasal prongs assembly, protruding chin cup and supporting headgear assembly (subject to separate disclosures), can be as small as approximately 96 mm by 101 mm by 73 mm.

Prior wearable CPAP devices mounted on the head or face of a user have been unsuccessful primarily because of the amount of noise and vibration transmitted directly to the skull of the user. They were also heavy and uncomfortable to wear. The new invention described herein provides the following advantages:
1) By mounting a small, light blower device on the user's chin, the present invention minimizes direct transmission of noise and vibration to the user's skull and ears. It also facilitates a short connection between the device and the user's nose and/or mouth without the need for a longer path from the blower that can dislodge the breathing interface as the user moves. The use of a chin cup with a cushioning lining provides a comfortable fit during extended (e.g., overnight) periods of wear and further reduces noise and vibration transmission to the user. It also enhance stability of the device on the user's head. The compact size and low weight of the systems described herein allow the system to be stable on the chin and not interfere with a user sleeping on his/her side or impact the user's neck or chest.
2) By using a high efficiency, compact blower and an extremely compact and efficient noise reduction system, without any noise absorbing acoustic foam, the device can be placed close to a user and bed partner without appreciable annoyance from excessive noise. The noise reduction elements described herein include:
   (a) An inlet air cover with multiple holes to minimize air resistance and provide some initial smoothing of the air flowing into the device. An optional conventional mesh air filter can be added anterior or posterior to this inlet air cover.
   (b) The inlet air cover is at the outer end of the inlet air chamber which has approximate cross-sectional dimensions of about 19 to 22 mm by about 23 to 24 mm, with rounded corners at the base of the airflow channel. Beyond the inlet air cover there is a resonant chamber of 0.1 to about 12 mm in length followed by a first unique honeycomb air straightener about 19 to 25 mm in length. In exemplary embodiments this honeycomb device has 6 or 7 hexagonal holes through it arranged in a hexagonal pattern with or without a central hole. The across flats dimension of the hexagonal holes is approximately 3 mm. Following this first honeycomb there is a second resonant chamber about 0.1 to 18 mm in length followed by a second honeycomb air straightener. Preferably the hexagonal channels in the first and second honeycomb are substantially “in line”, meaning sharing a common axis along their length axis. This second honeycomb releases its airflow into an elliptical cross-sectioned resonant chamber. The air is then transferred through an optional third air straightener before being transferred to the large upper housing volume outside and above the blower inlet hole.
   (c) The blower inlet hole is typically about 20 mm in diameter and has an air diffusing/smoothing cone at its center. A plastic or preferably rubber sleeve is inserted into the blower inlet hole to aid in smoothing the airflow into the blower. This sleeve is approximately 8 mm long with a wall thickness of about 3 mm and can be thinner and tapered into the blower inlet hole.
   (d) In one embodiment, an inner connecting sleeve has a friction fit and is prevented from protruding too far into the blower outlet hole by a blower outlet spacer approximately 5 mm long. This spacer is designed to keep the blower outlet sleeve separated from the outermost edge of the blower vane housing and the connecting rubber sleeve also functions to correctly orient the blower in the housing, and prevent it from rotating and moving. In a more compact embodiment, a bulbous blower outlet connector (described later) may be used to connect the blower outlet to the housing outlet.
   (e) A rubber shock-absorber mounting for the blower which is molded to fit the blower housing cavity. This rubber mounting is preferably made from 60 durometer biocompatible silicone This blower mounting allows easy and efficient assembly of the blower into the housing without screws, preventing movement of the blower when assembled and reducing transmission of vibrations from the blower to the housing (and user) and from the housing to the blower, and hence prevent damage/misalignment of the fragile blower bearings. The molded mounting has a protruding lip designed to seat into a corresponding groove on the blower and ideally be glued together with suitable adhesives.

The system housing contains a small CPAP blower, which is preferably about 51 mm in diameter by about 33 mm in height, drive and control electronics for the blower. A compact air inlet path from outside the device to the inlet hole of the blower that contains a set of defined resonant chambers and unique air straightening elements that provide a radiated blower noise of less than 32 dBA at 1 meter when operating at 10 cm H₂O pressure through a CPAP face mask.

In certain embodiments, the system comprises a removable rechargeable battery pack (e.g., containing four cylindrical 3600 mAh rechargeable batteries or 3 or more 5000 mAh rechargeable batteries) which provides electrical power to the drive control electronics and the blower. The batteries may be charged or supported in a wired or wireless manner. Alternatively, or together with the battery pack, the system comprises a connection to mains power which can provide electrical power to the drive control electronics and the blower, and/or provide electrical power for charging the batteries. In the case that there is no battery pack, the use of mains power or an external battery can provide a much lighter wearable device, albeit providing a system which is not entirely body worn due to the wired power connection.

Bracket 1, also referred to herein as a “chin cup,” further isolates the blower noise and vibration from the wearer, is used to mount the system on the user. The chin cup includes a resilient insert that provides comfort during extended wear and a secure fit for users with different chin sizes/shapes and chin positions. The chin cup insert has a rolling edge in contact with the wearer's chin (like a CPAP face mask) to avoid causing discomfort and red marks from pressure point edges of conventional pads. The insert is preferably made from biocompatible silicone and uses push fit pins on its base to ensure correct orientation in the chin cup holder as well as easy assembly/disassembly for cleaning. The chin cup and/or device housing may also uniquely contain biometric sensors in near or complete contact with the user to monitor vital parameters such as pulse-oximetry, breathing/other body sounds, body position/movement, temperature and so on. The chin cup insert is preferably of 50 to 75 durometer (A) with a flange wall thickness of approximately 1 to 2 mm. The chin cup insert is typically at least 6 mm high with a flange typically at least 6 mm wide. The elliptical hole in which the chin is inserted is typically 35 to 60 mm long and 20 to 30 mm wide. This central hole allows ventilation and cooling of the chin area which would not be feasible with a solid contact area. This chin cup design is readily applicable to other applications requiring lengthy contact times with a user such as for securing helmets and so on.

The construction can be manufactured using plastic injection molded parts which maintain accurate dimensions which are easily assembled. The system is thermally and electrically efficient with low noise and with low resistance to airflow, providing a current draw that increases from about 0.28 Amps to about 0.33 Amps at an inlet voltage of 14.8 V or 0.35 to 0.41 Amps at an input voltage of 11.1V when the noise abatement measures are added to the blower, and operating at 10 cm H₂O outlet pressure through a PAP face mask. The device can be used for over 10 hours at a continuous 10 cm H₂O pressure.

This system design described herein lends itself to other applications requiring small size, low noise, light weight, low heat production and low power consumption provided by batteries. One application would be in hazardous environment breathing equipment requiring powered airflow through filtering/cleansing media. Another application could be in high filtration allergy masks for acutely sensitive people. Other wearable applications would include devices for pumping fluids to advantageously compress or decompress parts of the body. While depicted herein as a chin-mounted system, the systems of the present invention could also be incorporated into a respiratory face mask, worn on top of the head, or on a user's chest or shoulder. In configurations where the face mask is not integral to the housing, the blower could, for example, be connected to a respiratory face/nasal mask or nasal prongs via tubing or other means. But by locating such a wearable blower device close to the user's face the use of uncomfortable, long, annoying and high resistance connection hoses can be avoided.

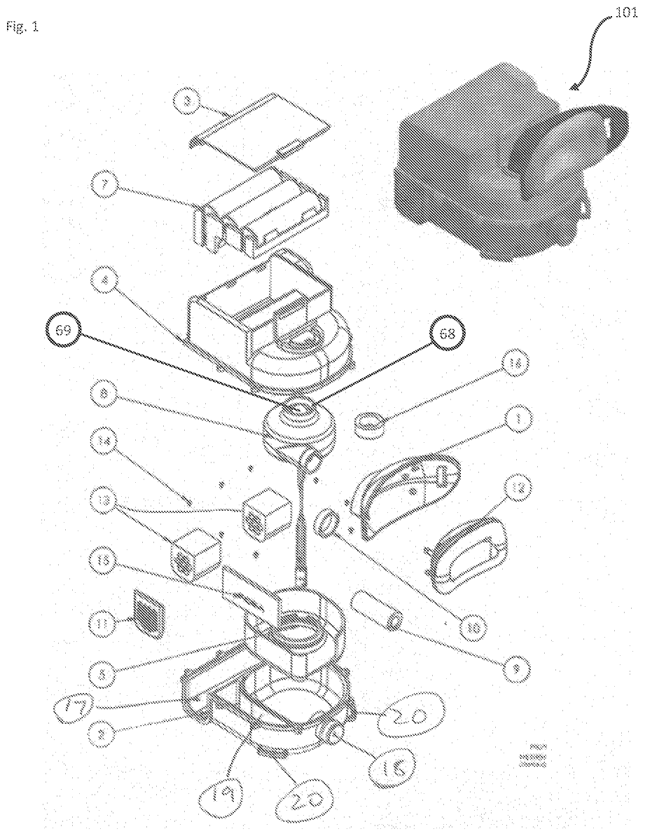

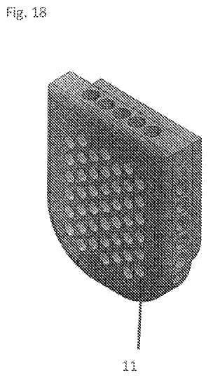

FIG. 1 is an exploded view of an exemplary PAP system 100 of the invention. A flow generator comprises a housing formed from a lower enclosure 2 and an upper enclosure 4. A blower 8 contains an impeller and a motor and is located within a resilient cushioning liner 5 within the housing. A battery power supply 7 positions within a battery compartment within the upper enclosure 4 which closes with lid 3. 17 is the location of the ambient air inlet. The blower inlet hole 68 has an air diffusing/smoothing cone 69 at its center. The outlet of the blower 8 has a blower outlet spacer 10 which properly locates the blower outlet sleeve 9 with respect to the blower's impeller outlet and this sleeve acts to properly locate the blower with respect to the main housing outlet 18. The blower inlet hole 26 contains an air smoothing inlet sleeve 16. The air inlet to the housing is protected with cover 11. Cover 11 may comprise air openings on a single surface, or the air openings may be on more than one surface of cover 11 as depicted in FIG. 18. Flow straighteners 13 are positioned in the air flow path between the air inlet and the blower as described hereinafter. As depicted, each air straightener has 7 holes, which may be of any shape but are preferably hexagonal. The number of holes on each air straightener can be the same or different relative to another air straightener. For example, the air straightener proximal to the ambient air inlet 17 A rigid or semi-rigid bracket 1 projects from the flow generator and is adapted to support and orient the flow generator on the mental protuberance of the wearer's mandible. A flexible chin support 12 inserts into bracket 1 to provide a soft interface on the mandible. The housing is held together with fasteners 14. A circuit board 15 providing control electronics for the system is housed in a compartment 19 within the housing. 20 are optional lugs for attaching a supporting headgear.

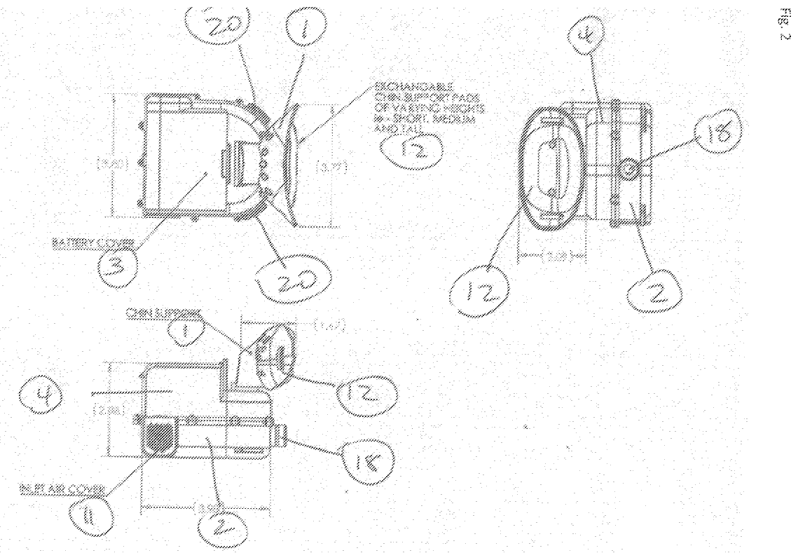

FIG. 2 provides various elevation views of the exemplary PAP system of the invention shown in FIG. 1 in assembled form.

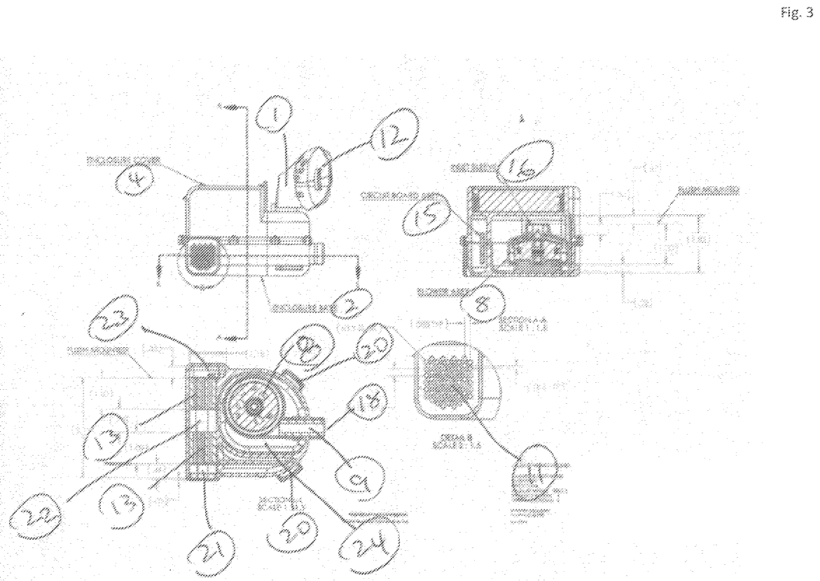

FIG. 3 depicts various cross-section views through the exemplary PAP system 101 of the invention shown in FIG. 1 in assembled form. All dimensions shown are in inches and are exemplary in nature only.

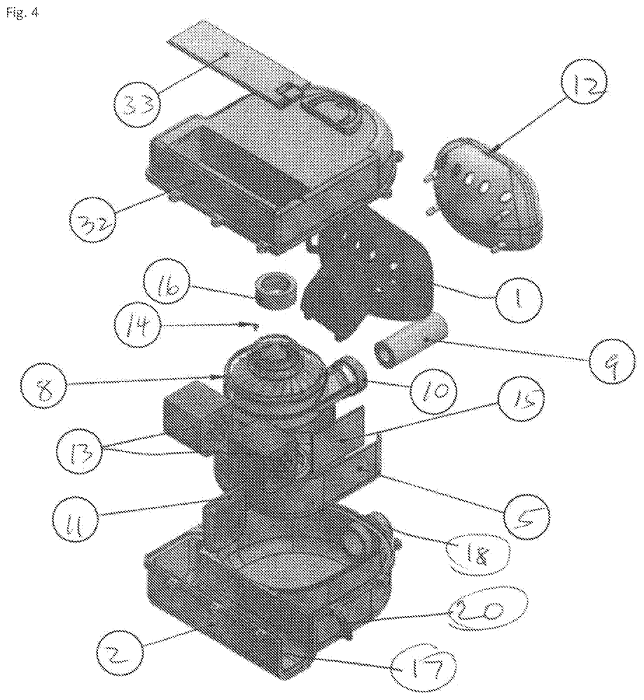

FIG. 4 is an exploded view of a second exemplary PAP system 101 of the invention. A flow generator comprises a housing formed from a lower enclosure 2 and an upper enclosure 32. A blower 8 contains an impeller and a motor and is located within a resilient cushioning liner 5 within the housing. The outlet of the blower 8 has a blower outlet spacer 10 which properly locates the blower outlet sleeve 9 with respect to the blower's impeller outlet and this sleeve acts to properly locate the blower with respect to the main housing outlet 18. The blower inlet hole 26 contains an air smoothing inlet sleeve 16. The air inlet to the housing is protected with cover 11. Flow straighteners 13 are positioned in the air flow path between the air inlet and the blower as described hereinafter. A rigid or semi-rigid bracket 1 projects from the flow generator and is adapted to support and orient the flow generator on the mental protuberance of the wearer's mandible. A flexible chin support 12 inserts into bracket 1 to provide a soft interface on the mandible. The housing is held together with fasteners 14. A circuit board 15 providing control electronics for the system is housed in a compartment 19 within the housing. 17 is the location of the ambient air inlet. 20 are optional lugs for attaching a supporting headgear. In this embodiment, a battery compartment within the upper enclosure is not included. Rather, a wired connection (e.g., a power cord, not shown) connects the electronics and blower to mains electrical power, either directly or through a low voltage transformer that sits external to the PAP system. Rather, an enclosure cover 32 comprises an opening for access to the system electronics is protected by cover 33.

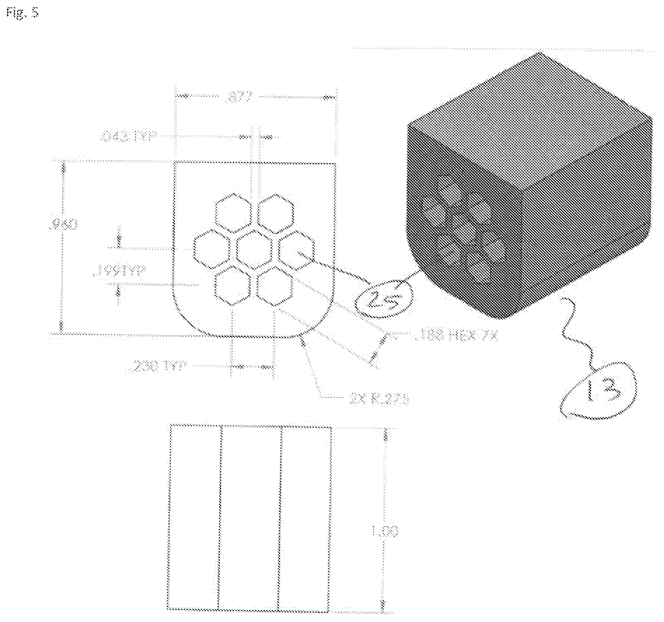

FIG. 5 depicts an exemplary flow straightener 13 as depicted in the exemplary PAP system of the invention shown in FIGS. 1 and 3 as a three-dimensional structure. Face and side elevations are shown. All dimensions shown are in inches and are exemplary in nature only. As depicted, the array of parallel air passages 25 are of substantially hexagonal shape in cross section and are arranged in the form of a hexagonal array. This is not meant to be limiting, as the air passages may be of any cross sectional shape, including but not limited to substantially circular, oval, polygonal, square, rectangular, etc., and may be arranged in any pattern through the flow straightener. By “substantially” with regard to shape is meant to indicate that the shape need not be a perfect hexagon, circle, oval, polygon, square, rectangle, etc., provided that the skilled artisan would recognize the shape as approximating one of these forms. A “flow straightener,” sometimes called a honeycomb, is a device used to straighten air flow in an air passage. Placed along the axis of main air stream, these devices minimize the lateral velocity components caused by swirling motion in the air flow during entry, thereby reducing turbulence and noise. While depicted here as a tube type of flow straightener, other flow straightener types such as fins and folded vanes are contemplated for use in the invention.

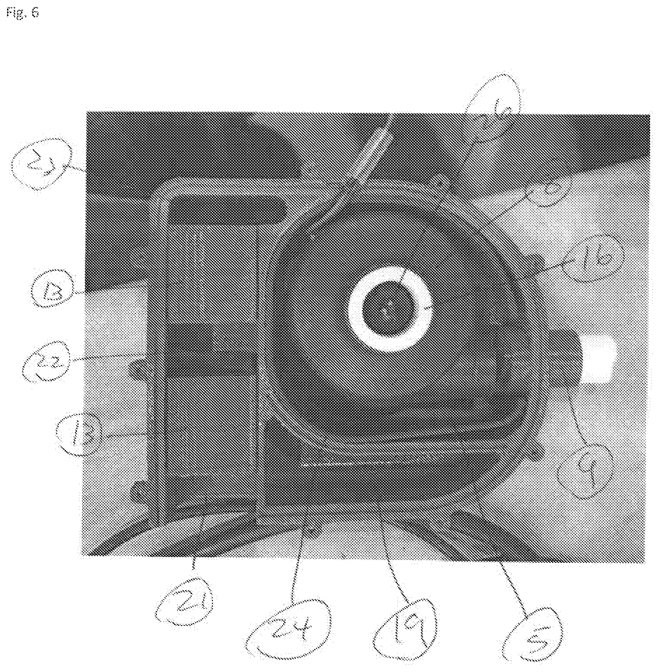

As described herein and shown in FIGS. 3 and 6, the exemplary PAP systems comprise at least a first and a second flow straightener 13 that are separated by an enclosed chamber 22. Preferably the pattern of arrangement of the air passages 25 are substantially coincident between the two flow straighteners (i.e., for each air passage in the first flow straightener, a corresponding air passage is provided in the second flow straightener such that each shares a common linear central axis).

FIG. 6 depicts the interior of the lower enclosure 2 with upper enclosure 4 removed, showing the air path taken from air inlet aperture to the air outlet aperture. Ambient air is drawn into the air inlet aperture and into a first chamber 21 proximal thereto. This chamber has a substantially rectangular cuboid shape and defining a void volume v1. By “substantially rectangular cuboid shape” is meant that the air chamber if in the form of a right rectangular prism, but may have one or more chamfered or radiused corners rather than sharp 90° corners. By “void volume” is meant the air-filled space enclosed by the walls of the chamber.

The air travels from first chamber 21 along a linear path through the first air straightener 13 and into a second chamber 22 which is again a substantially rectangular cuboid shape. The peripheral walls of the air straightener 13 that contact the lower enclosure 2 are constructed so that the exterior thereof fit the shape of the enclosure walls so that air straightener 13 may be sealed in an airtight manner into the lower enclosure 2. This first air straightener 13 comprises an array of parallel air passages as described herein. The air passages define a void volume of air straightener 13, meaning the portion of the air straightener through which the air may pass to reach the second chamber 22. A second air straightener 13 that is substantially identical to the first air straightener 13 follows the second chamber 22. Air travels through this second air straightener and into a third chamber 23. As depicted in FIG. 5, this third chamber has a radiused end that extends further laterally than the initial flow path. This third chamber 23 directs the air approximately 90° from the initial flow path and into a fourth chamber 24 which is formed between the lower enclosure 2 and upper enclosure 4 and houses the blower 8. The impeller of the blower receives this air in the fourth chamber through a blower air inlet opening 26 at the top of the blower. A resilient cushioning liner 5 isolates the blower from the lower enclosure 2 to reduce vibrations translated to the user and also to the blower itself. 16 and 9 are respectively the blower inlet and outlet sleeves.

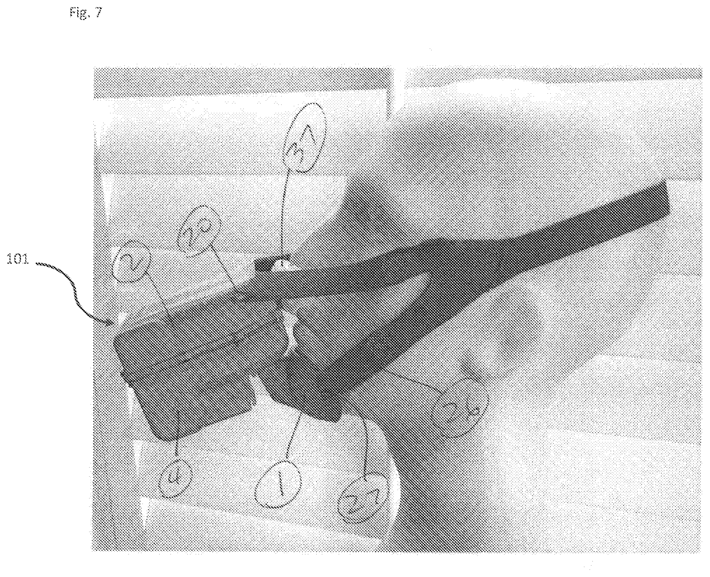

FIG. 7 depicts the exemplary PAP system 101 of the invention as worn on a user. As shown, the lower enclosure 2 is at the top of the system, with upper enclosure 4 underneath. The bracket 1 (“chin cup”) as shown is adapted to support and orient the flow generator on the mental protuberance of the individual's mandible. A pair of slots 20 are positioned on opposite sides of the housing, and a second pair of slots 27 are positioned on opposite sides of the bracket. One or more flexible head straps 26 couple to the slots 20 and 27 and engage the head of the individual to secure the device on the individual's mental protuberance. The patient interface 37 in this embodiment is depicted as a pair of nasal pillows adapted for at least partial insertion into the nares of the individual and in fluid communication with the air outlet aperture 18 in the housing. This is not meant to be limiting, and the patient interface may be any face mask known in the art.

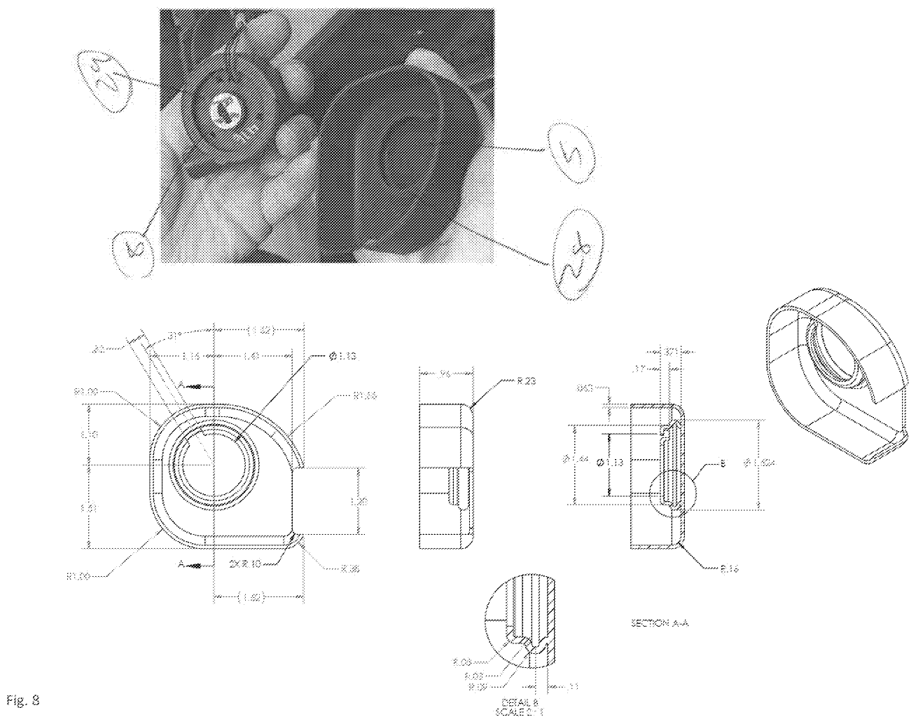

FIG. 8 depicts the resilient cushioning liner 5 in three dimensions and in drawings depicting exemplary dimensions in inches. A raised retaining feature 28 is integrally or unitarily formed on a surface of the resilient liner. As shown, blower 8 comprises a recessed portion 29 which seats onto the raised retaining feature 28.

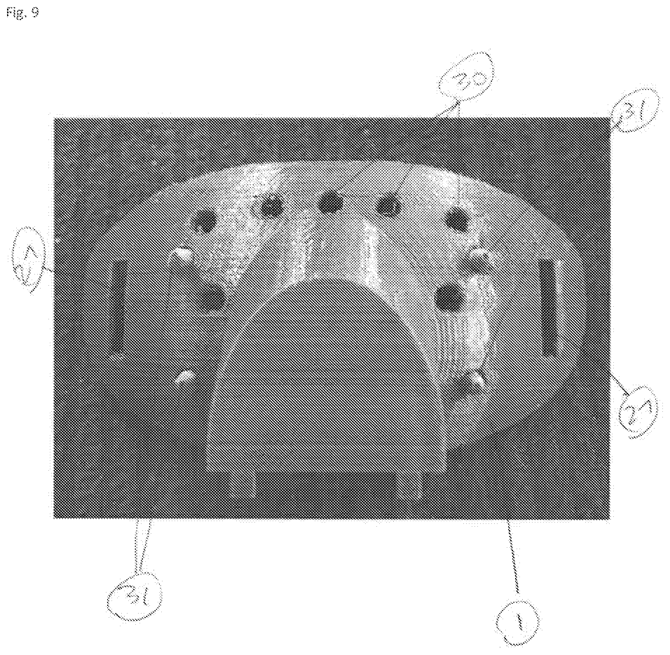

FIG. 9 depicts the back side (i.e., facing away from the wearer) of the chin cup 1, showing ventilation holes 30, slots 27, and push fit pins 31 which are part of, and act to retain, the chin cup insert 12. Optionally, additional slots 20 are provided on the lower enclosure 2 to provide additional anchor points for head straps 26.

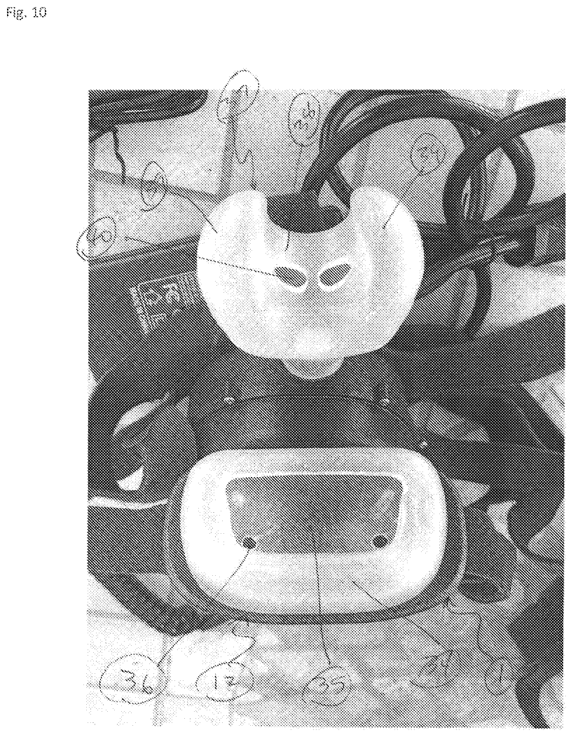

FIG. 10 depicts the PAP system 101 of the invention from the aspect facing the wearer. At the bottom, chin cup insert 12 that provides comfort during extended wear and a secure fit for users with different chin sizes/shapes and chin positions. The chin cup insert has a rolled edge 34 in contact with the wearer's chin (like a CPAP face mask), and a hollow that receives the bony protuberance of the chin. This structure helps to avoid causing discomfort and red marks from pressure point edges of conventional pads. Vent holes 36 act to provide temperature and humidity control within insert 12. Insert 12 is preferably made from a compliant and biocompatible material such as silicone and comprises push fit pins 31 on the surface in contact with the chin cup holder to ensure correct orientation in the chin cup insert as well as easy assembly/disassembly for cleaning. The chin cup insert 12 and/or device housing may also contain biometric sensors in near or complete contact with the user to monitor vital parameters such as pulse-oximetry, breathing/other body sounds, body position/movement, apnea/hypopnea detection, cardiac parameters, temperature, and so on. The chin cup insert 12 is preferably of 50 to 75 A durometer with a flange wall thickness of approximately 1 to 2 mm. The chin cup insert is typically at least 6 mm high with a flange typically at least 6 mm wide. The hole in which the chin is inserted is typically 35 to 60 mm long and 20 to 30 mm wide. These parameters can be adjusted as necessary by the artisan depending upon the anatomy of the wearer.

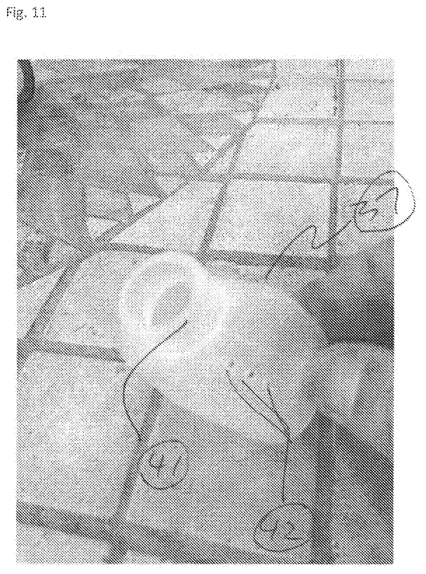

During operation, the PAP system creates positive air pressure through patient interface 37. As depicted in FIGS. 10 and 11, the patient interface 37 is a resilient hollow body comprising an inner chamber that acts as a resonator to dampen breathing sounds and noise from the PAP system. The resilient hollow body forms a roughly “C”-shaped form having a pair of lateral cushions, or “leaflets,” 39 on each side of recess 38. The patient's nose is positioned within recess 38 and between the lateral leaflets. By sealing to the patient's nose in this manner, the patient is spared the discomfort of having the patient interface insert into the nares of the patient as does a nasal pillow. Ring 41 provides a friction fit to main housing outlet 18. Air outlet holes 40 direct airflow from the PAP system towards the nares. The leaflets inhibit lateral movement of the face mask, and the near flat surface below and proximal to the nares is oriented at an appropriate angle so as not to leak when mounted on the patient.

In certain embodiments, pressurized air is delivered to a patient through patient interface 37 at a working pressure ranging from approximately 2 cm H₂O to approximately 30 cm H₂O above atmospheric pressure at the point of use, although any appropriate pressure may be used. Exhaust ports in the form of holes 42 are designed to prevent carbon dioxide build-up in the face mask and may be of appropriate configurations to reduce the exhaust noise. During inspiration, air from the PAP system that has accumulated at the working pressure in the hollow chamber is inhaled. During expiration, exhaled gas of the approximate volume of the patient interface 37 is deposited in the mask, with the rest exiting through the vent holes 42 or through the blower 8 itself. The continuous flow of air from the PAP system can also wash exhaled air out the patient interface 37 through these exhaust ports before the next inspiration.

Conventional PAP interfaces try to be as low profile and unobtrusive as possible and are held in place by directly attached headgear or straps and are connected to the CPAP unit by a long hose or hoses. Conversely, patient interface 37 is held in place directly by an upward force from the chin-mounted wearable PAP unit and uses no connection hose. Eliminating the high resistance connecting hose reduces the breathing resistance on exhalation through to the blower, making breathing more comfortable. Hose connections and changes in air flow direction and cross-sectional areas between a conventional CPAP unit and the user's face mask also provide discontinuities that affect the smooth flow of air between the CPAP unit and the face mask, and so result in additional sources of noise. By eliminating the connecting hose and other discontinuities, these additional noise sources are also eliminated reducing noise transmitted to both the user and the user's bed partner. Patient interface 37 has minimal abrupt geometry changes in the airflow path to minimize turbulence and noise, and is quieter than a conventional interface. This allows one embodiment of the interface design with vent holes 42 to be at least 2 dBA quieter than a conventional PAP interface. Also, when a compressible bellows section similar to that found in nasal pillows was inserted between the nares and the PAP unit of the invention, the interface was noisier than the smooth bulbous design of patient interface 37. It can be recognized that the interface can be larger or smaller as required to fit different nose sizes/shapes and accommodate different distances between the PAP system and a user's nose. As well as dissipating noise, the compliance of patient interface 37 acts as a compressible compliance buffer between the nose and the PAP system, so as to avoid the user's nose contacting the hard housing surfaces. To ensure comfort, fit, seal and compliance, the interface is preferably made from a 40 to 70 durometer biocompatible silicone (ideally 50 to 60) but higher and lower durometers and different materials may also be substituted.

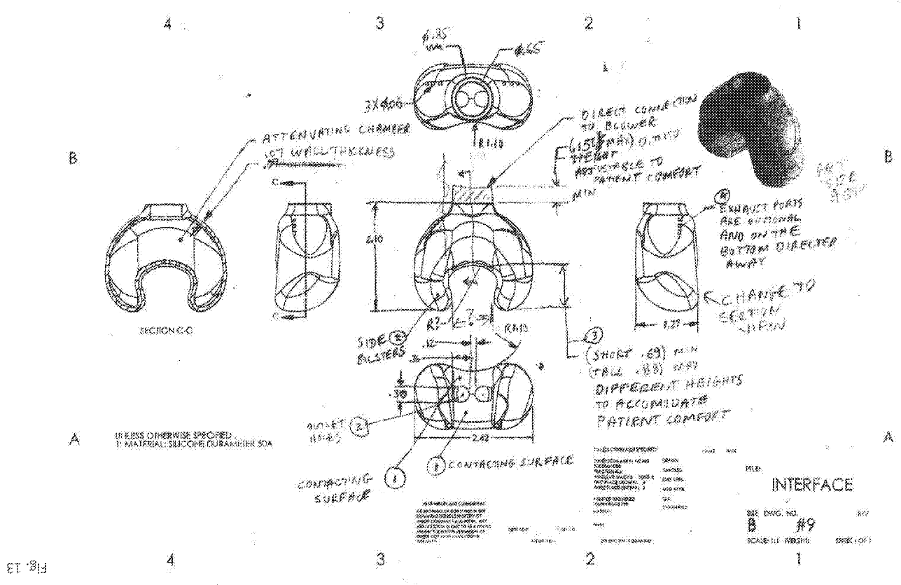

A detailed drawing of one example of patient interface 37 is provided in FIG. 13. To enhance comfort to the user as the interface is pushed upwards onto the user's nares, the contacting surface 38 is preferably broad and flat with one or two outlet holes 40 directing the airflow into the nares. The interface also has the lateral leaflets 39 to stop sideways movement of the interface relative to the nares and cause misalignment of the outlet holes 40 with the nares. In certain embodiments, the height of these leaflets relative to the flat area under the nares is between 12 and 40 mm to reach the nasal bone area and the bolsters are appropriately compliant to be comfortable, ideally using thin walled sections (typically 1.5 to 2.5 mm thick). The patient interface 37 can be molded in one piece using complex Liquid Silicone Rubber molding techniques or as a simpler two piece molding.

The exhaust holes 42 are preferably located on the bottom of the interface to exhaust air away from the user's face and not towards a bed partner. This location helps to minimize turbulence and noise in the interface and lines up directly with the exhaled air from the user. Exhaust holes 42 can be approximately 1.5 to 2.0 mm in diameter, but this arrangement and hole size can be varied as appropriate using known art to give the desired air exhaust flow and to minimize exhaust noise.

If humidification of the delivered air is required, a conventional hygroscopic condenser humidifier heat and moisture exchanger element can be inserted between the PAP unit blower and the patient's nares.

Up to 30% of current PAP users require a full-face or nose-mouth interface to deal with air/pressure leaks through their mouth. This is mitigated somewhat by the use of a head strap system to pull the PAP unit up on the user's chin, thereby assisting the user's mouth to stay shut and not leak air/pressure. If necessary, mouth leaks could be further eliminated by using an integrated nose/mouth interface instead of the nose-only interface described above.

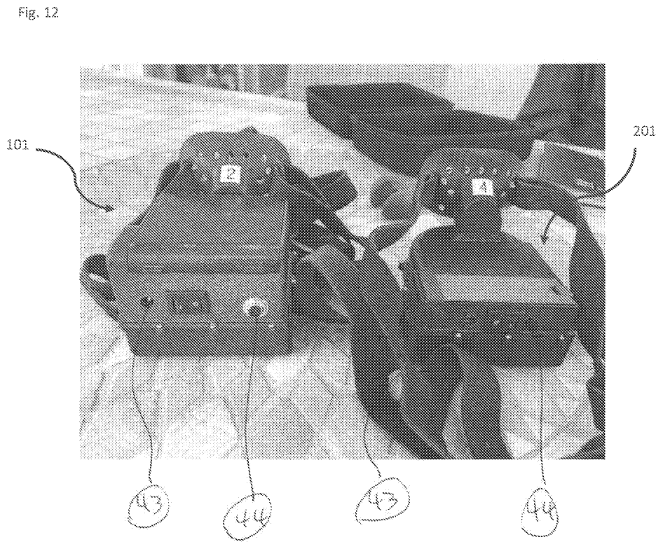

As discussed above, the energy supply for the PAP system may be internal to the body of the system (e.g., in the form of a battery pack), or may be located externally to the device housing. In FIG. 12, device 101 (on the left) comprises the internal battery, while the device 201 (on the right) is configured to run on mains power or an external battery. This external battery can be less limited by size and weight. Each device is pictured with a power jack 43 and a pump speed adjustment 44. Elimination of the battery from the body of the PAP system can result in the chin-worn portions of the PAP system being at least 50% lighter (0.2 kg vs 0.5 kg) and approximately 50% smaller. Because it is less limited by battery life, the PAP system without an internal battery is able to operate at higher working pressures for a longer time, have more power available for gathering and disseminating data from sensors embedded in the device, and allow the PAP unit to be used more comfortably on children and small adults. In certain embodiments, the external battery could be located on the patient's body (e.g. top of head) or somewhere convenient for the patient near their sleeping position (e.g. above the pillow) or removably attached to the chin cup.

Children in particular often suffer with unstable, poorly-fitting, large claustrophobic face masks tied to a heavy hose connecting the mask to a bedside PAP unit. The external power supply could be connected to the lighter wearable PAP unit 201 using a spiral cable like a coiled telephone handset cable. This would allow the cable to maintain a short unobtrusive length under many conditions but be readily and easily extendable to accommodate user movements and changes of sleeping position. Such cables are light and robust, and able to withstand long-term bending and stretching in use. This device configuration without an internal battery would also be useful in hazardous environment breathing equipment requiring high pressures for long periods of time.

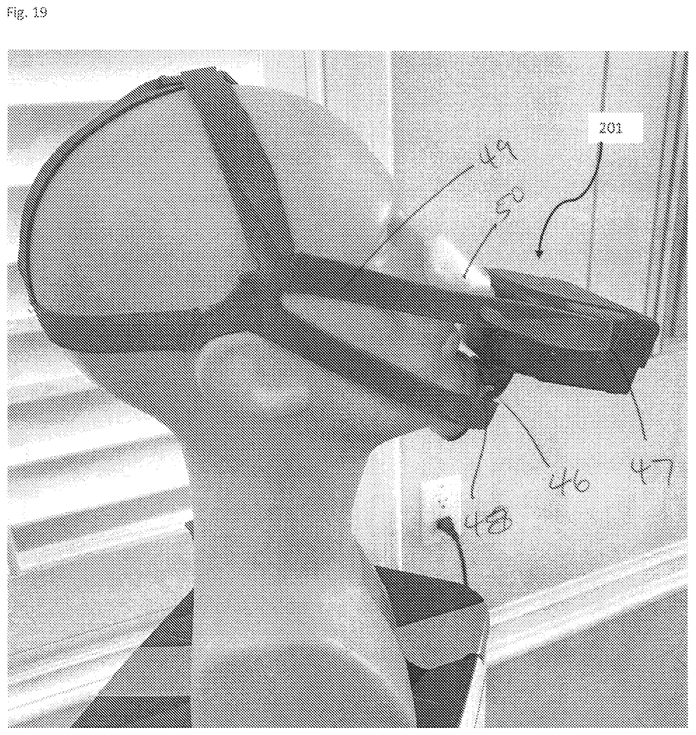

In an analogous manner to FIG. 7, FIG. 19 depicts the exemplary PAP device 201 of the invention, configured to operate with an external power supply (not shown), as worn on a user. The bracket 46 (“chin cup”) as shown is adapted to support and orient the flow generator on the mental protuberance of the individual's mandible. A pair of slots 47 are positioned on opposite sides of the housing, and a second pair of slots 48 are positioned on opposite sides of the bracket. One or more flexible head straps 49 couple to the slots 47 and 48 and engage the head of the individual to secure the device on the individual's mental protuberance. The patient interface 50 in this embodiment is depicted as a pair of nasal pillows adapted for at least partial insertion into the nares of the individual and in fluid communication with the air outlet aperture in the housing. This is not meant to be limiting, and the patient interface may be any face mask known in the art.

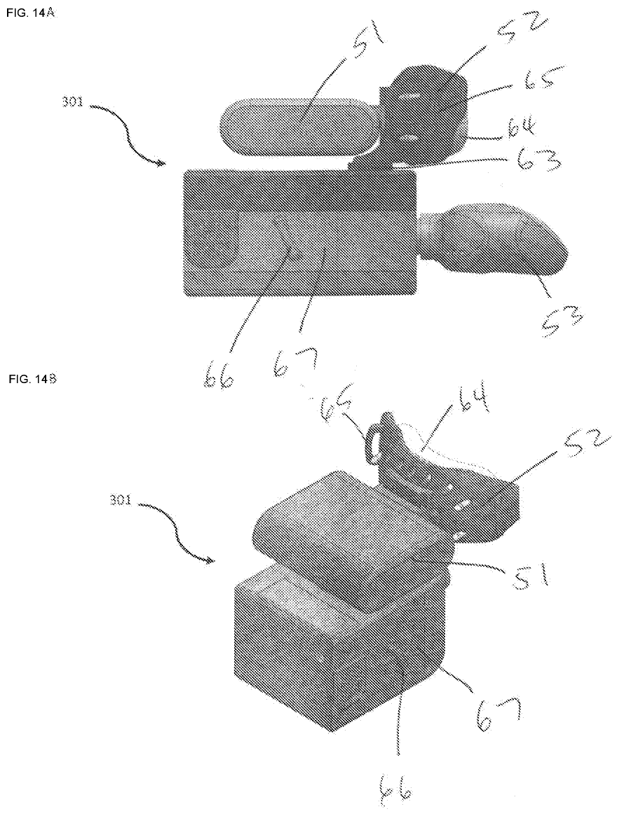

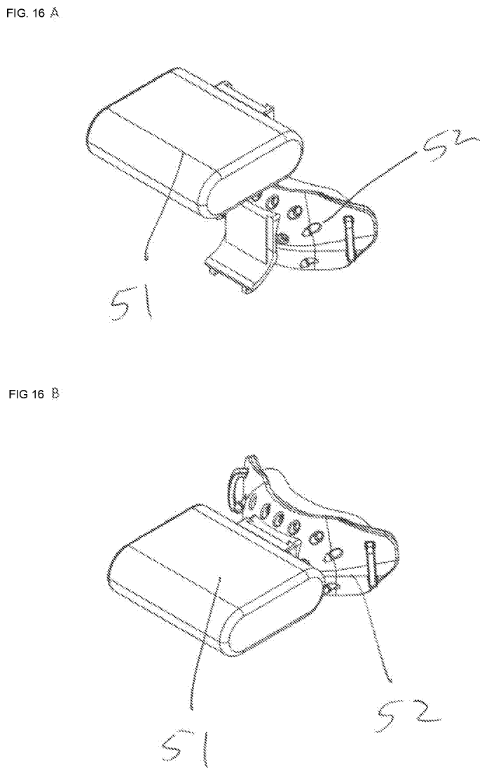

In still another exemplary embodiment, FIGS. 14 A and B depict two views of exemplary PAP device 301. In this embodiment, a power supply (e.g., battery pack) 51 is supported, not on the air pump housing, but instead on the bracket 52 (“chin cup”), with the bracket itself mounted on the flow generator. This embodiment provides less weight supported on the patient interface 53, and thus less torque is applied to the wearer's nose as the wearer moves during use. Positioning the weight of the battery pack in this manner may impart additional comfort and stability to the PAP device. The rigid or semi-rigid bracket 52 projects from the power supply 51 and is adapted to support and orient the power supply on the mental protuberance of the wearer's mandible. The flow generator 67 mates to the bracket at an anchor point 63 on. A flexible chin support 64 inserts into bracket 52 to provide a soft interface on the mandible. 65 and 66 are optional lugs for attaching a supporting headgear. The power supply can be attached in a sliding manner as shown in FIG. 16. FIG. 16A depicts the bracket 52 with the power supply 51 unmounted thereto, and FIG. 16B shows depicts the bracket 52 with the power supply 51 attached via the sliding fit. This friction-slide fit permits a certain amount of vertical adjustment of the power supply 51 relative to the chin cup so that the wearer can adjust the positon of the power supply for comfort as may be desired.

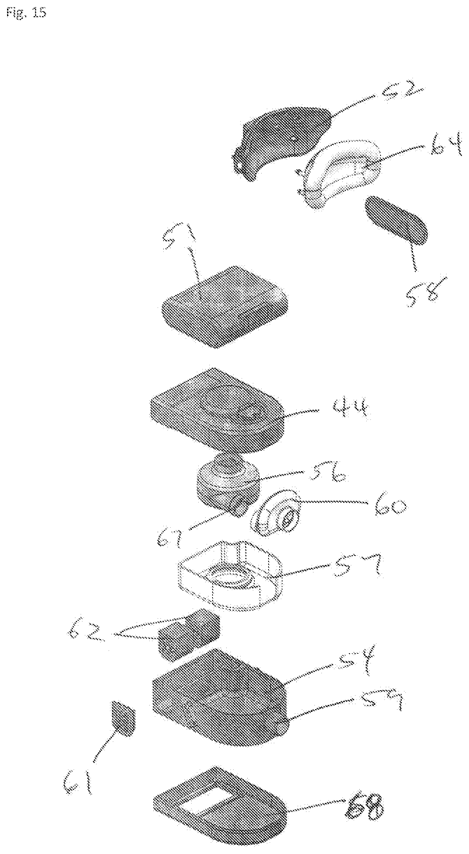

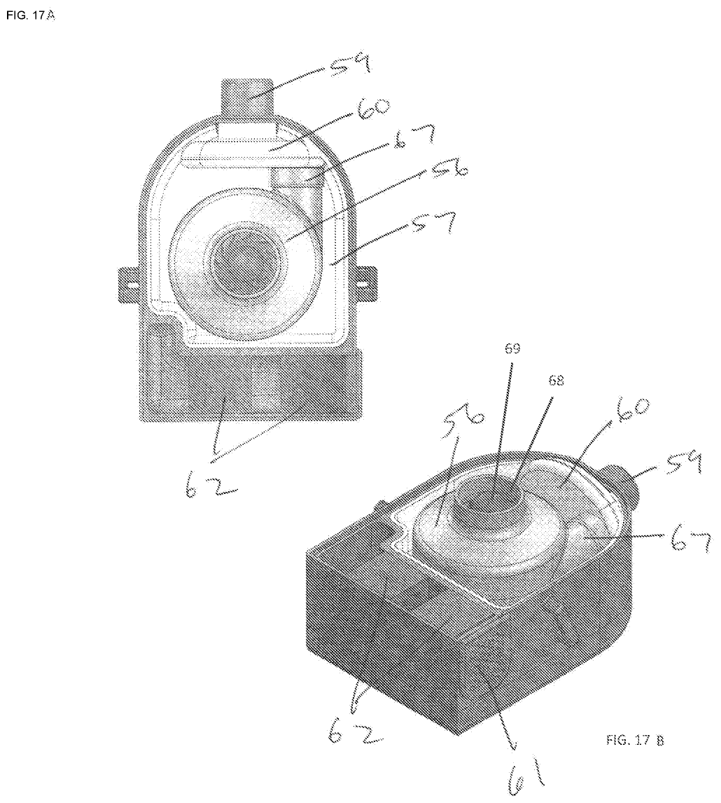

Exemplary PAP device 301 is shown in exploded form in FIG. 15 and partially assembled in FIGS. 17 A and B. A flow generator comprises a housing formed from a lower enclosure 54 and an upper enclosure 44. A blower 56 contains an impeller and a motor and is located within a resilient cushioning liner 57 within the housing. A battery power supply 51 closes with lid 58. The outlet 67 of the blower 56 connects to the main housing outlet 59 via a bulbous blower outlet connector 60. This bulbous blower outlet connector has an interior which is smooth, with as large a clearance as feasible and a rounded bulbous shape to minimize noise and air turbulence. An offset between the blower side and the housing side of the blower outlet connector 60 can permit more compact packaging within the housing. This is shown in more detail in FIG. 17. The air inlet to the housing is protected with cover 61. Flow straighteners 62 are positioned in the air flow path between the air inlet and the blower as described herein. A compartment for control electronics is formed by mating cover 68 to lower enclosure 54.

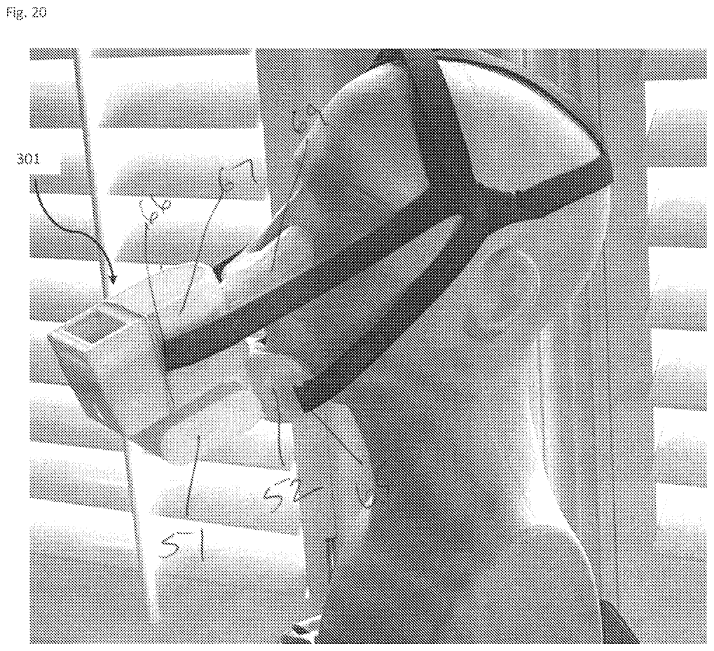

In an analogous manner to FIG. 7, FIG. 20 depicts the exemplary PAP device 301 of the invention, configured to operate with a removable battery power supply 51 supported by the bracket 52 (“chin cup”), as worn on a user. The bracket 52 as shown is adapted to support and orient the power supply 51 on the mental protuberance of the individual's mandible. The removable battery pack may slide into position on the chin cup and be able to move backwards and forwards to give a more comfortable position for the user, or alternatively use a hinge mechanism for attachment to the chin cup. The flow generator 67 sits on anchor point 63 (between 51 and 67). A pair of slots 65 are positioned on opposite sides of the housing, and a second pair of slots 66 are positioned on opposite sides of the bracket. One or more flexible head straps 67 couple to the slots 65 and 66 to engage the head of the individual to secure the device on the individual's mental protuberance. The patient interface 69 in this embodiment is depicted as a pair of nasal pillows adapted for at least partial insertion into the nares of the individual and in fluid communication with the air outlet aperture in the housing. This is not meant to be limiting, and the patient interface may be any face mask known in the art.

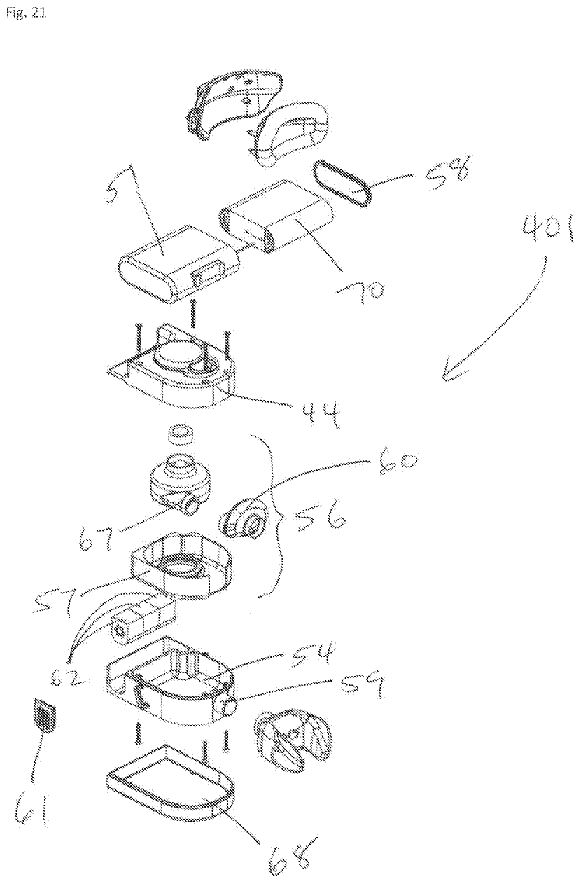

Exemplary PAP device 401 is shown in exploded form in FIG. 21 and partially assembled in FIGS. 17 A and B. A flow generator comprises a housing formed from a lower enclosure 54 and an upper enclosure 44. A blower 56 contains an impeller and a motor and is located within a resilient cushioning liner 57 within the housing. A battery power supply 51 closes with lid 58 to hold battery cells 70. The outlet 67 of the blower 56 connects to the main housing outlet 59 via a bulbous blower outlet connector 60. This bulbous blower outlet connector has an interior which is smooth, with as large a clearance as feasible and a rounded bulbous shape to minimize noise and air turbulence. An offset between the blower side and the housing side of the blower outlet connector 60 can permit more compact packaging within the housing. This is shown in more detail in FIG. 17. The air inlet to the housing is protected with cover 61. Three flow straighteners 62 are positioned in the air flow path between the air inlet and the blower as described herein, with a spacing between the flow straighteners of between 0.1 and 0.5 mm. Preferably the air channels in the three flow straighteners are in a 7-6-7 pattern as described herein. A compartment for control electronics is formed by mating cover 68 to lower enclosure 54.

### PREFERRED EMBODIMENTS

The following are preferred embodiments of the invention.

Embodiment 1. A flow generator adapted for use in a PAP system, comprising:
a housing;
an air inlet aperture formed in a wall of the housing configured to allow atmospheric air into the housing;
an air path within the housing in fluid communication with the air inlet aperture, comprising
   a first chamber proximal to the air inlet aperture having a substantially rectangular cuboid shape,
   a plurality of flow straighteners, each flow straightener comprising an array of substantially parallel air passages, wherein the first flow straightener in the plurality is downstream of, and in fluid communication with, the first chamber, wherein the remaining flow straightener in the plurality are in serial fluid communication with the first flow straightener, and wherein a chamber follows each flow straightener in the plurality, wherein the chamber is an unoccupied space in the air path, and
a blower chamber within the housing immediately downstream of, and in fluid communication with, the chamber following the last flow straightener in the plurality;
a blower within the blower chamber comprising an impeller and a motor; and
an air outlet aperture formed in a wall of the housing operably connected to the blower to receive air exiting an air output portion of the blower,
wherein the blower, when energized, is configured to draw atmospheric air into the inlet aperture and thereby generate an airflow through the air path and into the blower, and direct the airflow at greater than atmospheric pressure to the air outlet aperture

Embodiment 2. A flow generator according to embodiment 1, comprising:
a housing;
an air inlet aperture formed in a wall of the housing configured to allow atmospheric air into the housing;
an air path within the housing in fluid communication with the air inlet aperture, comprising
   a first chamber proximal to the air inlet aperture having a substantially rectangular cuboid shape,
   a first flow straightener comprising a first array of substantially parallel air passages downstream of, and in fluid communication with, the first chamber,
   a second chamber immediately downstream of, and in fluid communication with, the first air straightener having a substantially rectangular cuboid shape, and
   a second flow straightener comprising a second array of parallel air passages downstream of, and in fluid communication with, the second chamber,
a third chamber immediately downstream of, and in fluid communication with, the second air straightener, and
a fourth chamber within the housing immediately downstream of, and in fluid communication with, the third chamber;
a blower within the fourth chamber comprising an impeller and a motor; and
an air outlet aperture formed in a wall of the housing operably connected to the blower to receive air exiting an air output portion of the blower,
wherein the blower, when energized, is configured to draw atmospheric air into the inlet aperture and thereby generate an airflow through the air path and into the fourth chamber, and direct the airflow at greater than atmospheric pressure to the air outlet aperture.

Embodiment 3. A flow generator according to embodiment 1, comprising:
a housing;
an air inlet aperture formed in a wall of the housing configured to allow atmospheric air into the housing;
an air path within the housing in fluid communication with the air inlet aperture, comprising
   a first chamber proximal to the air inlet aperture having a substantially rectangular cuboid shape,
   a first, second, and third flow straightener comprising a first array of substantially parallel air passages, wherein the first flow straightener is downstream of, and in fluid communication with, the first chamber, the second flow straightener comprising a second array of parallel air passages is downstream of, and in fluid communication with the first flow straightener, and the third flow straightener comprising a third array of parallel air passages is downstream of, and in fluid communication with the second flow straightener,
   a chamber following each flow chamber, wherein the chamber is an unoccupied space in the air path, and
a blower chamber within the housing immediately downstream of, and in fluid communication with, the chamber following the third flow straightener;
a blower within the blower chamber comprising an impeller and a motor; and
an air outlet aperture formed in a wall of the housing operably connected to the blower to receive air exiting an air output portion of the blower,
wherein
the blower, when energized, is configured to draw atmospheric air into the inlet aperture and thereby generate an airflow through the air path and into the blower, and direct the airflow at greater than atmospheric pressure to the air outlet aperture.

Embodiment 4. A flow generator according to embodiment 3, wherein the spacing between the first and second flow straighteners and the spacing between the second and third flow straighteners are each independently between 0.1 and 0.3 mm, and
the first, second, and third flow straighteners together occupy between 75% and 99% of the volume between the air inlet aperture and the blower chamber, measured without accounting for the volume occupied by the array of parallel air passages in each flow straightener.

Embodiment 5. A flow generator according to embodiment 3 or 4, wherein the first flow straightener comprises seven air passages, the second flow straightener comprises six air passages, and the third flow straightener comprises seven air passages, resulting in a 7-6-7 pattern of preferably hexagonal air passages in the three flow straighteners, wherein one or more of the air passages in each flow straightener are substantially “in line” with corresponding air passages in an ajoining flow straightener, and at least one air passage in the “7” flow straightener is not in line with a corresponding air passage in the ajoining “6” flow straightener.

Embodiment 6. A flow generator according to one of embodiments 1-5, wherein each air passage in the plurality of flow straighteners are substantially identical in cross-section.

Embodiment 7. A flow generator according to one of embodiments 1-6, wherein each air passage in the plurality of flow straighteners are substantially hexagonal in cross-section.

Embodiment 8. A flow generator according to embodiment 2, having at least one of the following characteristics:
i) the volume of the first chamber is between 0.1 mm³ to 6,500 mm³,
ii) the volume of the second chamber is approximately 3,000 to 10,000 mm³,
iii) the volume of the third chamber ahead of an optional third air straightener is approximately 3,000 to 10,000 mm³,
iv) the volume of the final chamber connecting to the chamber containing the blower is approximately 4,000 to 14,000 mm³,
v) the length of each array of air passages in the air straightener elements is approximately 19 to 25 mm, and
vi) the cross-sectional area of each hexagonal flow passage in the first, second and optional third array of air passages is approximately 8 mm².

Embodiment 9. A flow generator according to embodiment 8 having each of characteristics i) through vi).

Embodiment 10. A flow generator according to one of embodiments 1-9, wherein one or more air passages in each of flow straightener in the plurality is in line with a corresponding air passage with the other flow straighteners in the plurality.

Embodiment 11. A flow generator according to one of embodiments 1-10, wherein the central axis of the air path through the plurality of flow straighteners is approximately linear, and the central axis of the air path from the chamber following the final flow straightener in the plurality to the blower chamber diverges from the central axis of the air path from the first flow chamber to the third flow chamber.

Embodiment 12. A flow generator according to embodiment 11 wherein the divergence is approximately 90° to the central axis of the air path through the plurality of flow straighteners.

Embodiment 13. A flow generator according to one of embodiments 1-12, wherein the airflow through the flow generator does not pass through a foam material between the air inlet aperture and the air output aperture.

Embodiment 14. A flow generator according to one of embodiments 1-13, wherein the blower chamber comprises a resilient liner, wherein the blower is adapted to seat in a raised retaining feature integrally or unitarily formed on a surface of the resilient liner.

Embodiment 15. A PAP system adapted for treatment of an individual having respiratory disease, sleep disordered breathing, or requiring breathing support, comprising:
a flow generator, comprising
a housing,
a blower comprising an impeller and a motor within the housing, and
a flow outlet within the housing, wherein the flow generator is adapted to provide a flow of pressurised breathable gas produced by the blower through the flow outlet;
a power supply operably connected to the flow generator and adapted to provide electrical energy to energize the blower;
a securement interface, comprising
a bracket projecting from the flow generator or the power supply, wherein the bracket is adapted to support and orient the flow generator or the power supply on the mental protuberance of the individual's mandible and to isolate the individual's cranial bones from noise and vibration generated during operation of the flow generator,
at least two slots on the bracket configured and arranged to be on opposite sides of individual's the mental protuberance,
a one or more flexible head straps adapted to couple to the at least two slots on the bracket and to engage the head of the individual to secure the bracket on the individual's the mental protuberance;
a patient interface, comprising
an interface portion in fluid communication with the flow outlet of the flow generator, and
a face mask adapted to administer airflow produced by the flow generator into an airway of the individual.

Embodiment 16. A PAP system according to embodiment 15, wherein the bracket projects from the flow generator and the power supply is integrated within the flow generator housing.

Embodiment 17. A PAP system according to embodiment 15, wherein the wherein the bracket projects from the power supply, and the power supply is attached to an external surface of the flow generator housing.

Embodiment 18. A PAP system according to embodiment 15, wherein the wherein the bracket projects from the flow generator, and the power supply is separate from the flow generator and connected by a cable providing an electrical connection between the power supply and the flow generator.

Embodiment 19. A PAP system according to embodiment 18, wherein the power supply operably connects to a mains power source.

Embodiment 20. A PAP system according to one of embodiments 15-19, wherein the face mask comprises a pair of nasal pillows adapted for at least partial insertion into the nares of the individual.

Embodiment 21. A PAP system according to one of embodiments 15-20, wherein the bracket projecting from the flow generator comprises a chin cup adapted to conform approximately to the shape of the mental protuberance, and a chin cup insert which is adapted to form a cushioned contact surface between the chin cup and the mental protuberance.

Embodiment 22. A PAP system according to one of embodiments 15-21, wherein the flow generator is according to one of embodiments 1-14.

Embodiment 23. A PAP system according to one of embodiments 15-22, wherein the power supply comprises a battery, and wherein the PAP system is configured to provide a continuous 10 cm H₂O pressure and 76 L/min flow rate for at least 10 hours.

Embodiment 24. A PAP system according to one of embodiments 15-23, wherein the PAP system weighs less than 0.5 kg in use.

Embodiment 25. A PAP system according to one of embodiments 15-24, wherein the face mask is in the form of a resonant chamber formed from a resilient material which embraces the nose without insertion into the nares.

Embodiment 26. A PAP system according to one of embodiments 15-25, wherein the face mask is in the form of a hollow body comprising an inner chamber and formed of a resilient material forming a c-shaped cavity configured to receive a patient's nose and to form a seal against the patient's nose, and one or more air outlet openings through the hollow body within the c-shaped cavity configured to direct air flow entering the hollow body from the PAP system towards the nares of the patient.

Embodiment 27. A face mask adapted for use with a PAP system, comprising:
a hollow body comprising an inner chamber and formed of a resilient material forming a c-shaped cavity configured to receive a patient's nose and to form a seal against the patient's nose;
a flow generator interface portion configured for fluid communication between an air flow outlet of the PAP system and the inner chamber of the hollow body; and
one or more air outlet openings through the hollow body within the c-shaped cavity configured to direct air flow entering the hollow body from the PAP system towards the nares of the patient.

Embodiment 28. A face mask according to embodiment 27, further comprising one or more vent holes through the hollow body configured to permit air to exhaust from the inner chamber into the atmosphere.

Embodiment 29. A method of treating sleep apnea, snoring, or another breathing disorder in an individual, comprising:
securing a PAP system of one of embodiments 15-26 to the individual by positioning the bracket projecting from the flow generator onto the mental protuberance of the individual's mandible, engaging the head straps to the head of the individual to maintain the flow generator on the mental protuberance, and positioning the face mask over an external airway passage of the individual; and
energizing the flow generator to administer airflow produced by the flow generator into the individual's airway via the face mask.

Embodiment 30. A PAP system adapted for treatment of an individual having respiratory disease, sleep disordered breathing, or requiring breathing support comprising a flow generator configured to engage onto the mental protuberance of the individual's mandible and be retained thereon by head straps.

Embodiment 31. A PAP interface comprising:
a bulbous, hollow chamber comprising an air inlet hole configured to operably connect to a PAP system to receive airflow produced thereby, a pair of leaflets which wrap around the exterior of an individual's nose and thereby position at least one air exit hole in the hollow chamber at each nares of the individual, and one or more vent holes, wherein the bulbous, hollow chamber is formed from a 40 to 70 durometer biocompatible material configured to provide a compressible compliance buffer between the nose and the PAP system.

### Example 1

The radiated sound pressure level and airflow produced by a prototype PAP system of the invention operating at 10 cm H₂O was measured at a distance of 1 m substantially as described in ISO 17510:2015 exhausting through a 0.179 inch plate orifice versus a commercially available ResMed Air Mini system is shown in Figure XXX.

Sound pressure values of a variety of objects are listed below.

| Object | Sound pressure dB(A) | Notes |
|---|---|---|
| Conversational speech | 60 | 1 m distance |
| Average home | 50 | |
| Quiet library | 40 | |
| Quiet bedroom at night | 30 | |
| Background in TV studio | 20 | |

### Example 2

The following table shows the flow performance of an embodiment of the invention shown in FIGS. 4 and 12 which used an external power source supplying either an 11.1 V or 14.8 V input voltage. The flow characteristics are similar to other FDA approved CPAP devices and can provide sufficient flow (over 40 LPM) in a simulation of a patient taking a rapid breath (represented by a plate orifice much larger than a 0.179 inch plate orifice, which represents the size of exhaust ports on a CPAP mask). Even with a high flow rate of 40 LPM the current draw was less than 0.36 Amps at pressures up to 10 cmH2O with a 14.8 V supply voltage and less than 0.46 Amps with an 11.1 V supply voltage. The tests were conducted at 1400 feet elevation at approximately 80 degrees Fahrenheit.

| | | | Actual | | |
|---|---|---|---|---|---|
| | | | Analyzer | | Orifice |
| Nominal | | Current, | Pressure | Flow, | Diameter, |
| Pressure | Voltage | Amps | (cmH2O) | LPM | inches |
| 04 cmH2O | 14.8 V | 0.120 | 4.01 | 17.71 | 0.179 |
| 04 cmH2O | 14.8 V | 0.154 | 3.97 | 29.68 | 0.200 |
| 04 cmH2O | 14.8 V | 0.161 | 4.00 | 30.13 | 0.240 |
| 04 cmH2O | 14.8 V | 0.178 | 4.02 | 34.63 | 0.268 |
| 04 cmH2O | 14.8 V | 0.214 | 4.01 | 41.29 | 0.303 |
| 04 cmH2O | 14.8 V | 0.092 | 3.99 | 0.00 | blocked |
| 06 cmH2O | 14.8 V | 0.164 | 6.04 | 21.52 | 0.179 |
| 06 cmH2O | 14.8 V | 0.185 | 6.03 | 26.87 | 0.200 |
| 06 cmH2O | 14.8 V | 0.230 | 6.03 | 36.47 | 0.240 |
| 06 cmH2O | 14.8 V | 0.262 | 6.03 | 42.10 | 0.268 |
| 06 cmH2O | 14.8 V | 0.347 | 5.97 | 53.85 | 0.303 |
| 06 cmH2O | 14.8 V | 0.115 | 6.03 | 0.00 | blocked |
| 08 cmH2O | 14.8 V | 0.216 | 7.97 | 24.73 | 0.179 |
| 08 cmH2O | 14.8 V | 0.240 | 8.02 | 30.52 | 0.200 |
| 08 cmH2O | 14.8 V | 0.302 | 7.97 | 41.74 | 0.240 |
| 08 cmH2O | 14.8 V | 0.350 | 8.02 | 48.49 | 0.268 |
| 08 cmH2O | 14.8 V | 0.474 | 7.99 | 61.94 | 0.303 |
| 08 cmH2O | 14.8 V | 0.145 | 8.03 | 0.00 | blocked |
| 10 cmH2O | 14.8 V | 0.275 | 10.01 | 27.86 | 0.179 |
| 10 cmH2O | 14.8 V | 0.314 | 10.03 | 34.29 | 0.200 |
| 10 cmH2O | 14.8 V | 0.398 | 10.01 | 46.86 | 0.240 |
| 10 cmH2O | 14.8 V | 0.459 | 10.04 | 53.97 | 0.268 |
| 10 cmH2O | 14.8 V | 0.577 | 10.03 | 65.24 | 0.303 |
| 10 cmH2O | 14.8 V | 0.173 | 10.03 | 0.00 | blocked |
| 12 cmH2O | 14.8 V | 0.318 | 11.98 | 30.22 | 0.179 |
| 12 cmH2O | 14.8 V | 0.368 | 12.00 | 37.60 | 0.200 |
| 12 cmH2O | 14.8 V | 0.476 | 11.99 | 50.97 | 0.240 |
| 12 cmH2O | 14.8 V | 0.559 | 11.98 | 59.00 | 0.268 |
| 12 cmH2O | 14.8 V | 0.795 | 12.00 | 76.00 | 0.303 |
| 12 cmH2O | 14.8 V | 0.199 | 12.03 | 0.00 | blocked |
| 04 cmH2O | 11.1 V | 0.148 | 3.97 | 17.61 | 0.179 |
| 04 cmH2O | 11.1 V | 0.162 | 4.03 | 21.92 | 0.200 |
| 04 cmH2O | 11.1 V | 0.197 | 4.01 | 29.65 | 0.240 |
| 04 cmH2O | 11.1 V | 0.216 | 3.95 | 34.16 | 0.268 |
| 04 cmH2O | 11.1 V | 0.262 | 3.99 | 41.30 | 0.303 |
| 04 cmH2O | 11.1 V | 0.108 | 4.00 | 0.00 | blocked |
| 06 cmH2O | 11.1 V | 0.210 | 6.03 | 22.14 | 0.179 |
| 06 cmH2O | 11.1 V | 0.231 | 6.02 | 26.74 | 0.200 |
| 06 cmH2O | 11.1 V | 0.284 | 5.97 | 36.21 | 0.240 |
| 06 cmH2O | 11.1 V | 0.327 | 6.00 | 41.79 | 0.268 |
| 06 cmH2O | 11.1 V | 0.410 | 6.03 | 50.59 | 0.303 |
| 06 cmH2O | 11.1 V | 0.142 | 6.00 | 0.00 | blocked |
| 08 cmH2O | 11.1 V | 0.272 | 8.02 | 24.89 | 0.179 |
| 08 cmH2O | 11.1 V | 0.307 | 8.00 | 30.77 | 0.200 |
| 08 cmH2O | 11.1 V | 0.389 | 8.03 | 41.92 | 0.240 |
| 08 cmH2O | 11.1 V | 0.452 | 8.02 | 48.26 | 0.268 |
| 08 cmH2O | 11.1 V | 0.575 | 8.02 | 58.59 | 0.303 |
| 08 cmH2O | 11.1 V | 0.178 | 8.03 | 0.00 | blocked |
| 10 cmH2O | 11.1 V | 0.348 | 10.08 | 27.85 | 0.179 |
| 10 cmH2O | 11.1 V | 0.394 | 9.97 | 34.27 | 0.200 |
| 10 cmH2O | 11.1 V | 0.510 | 9.96 | 46.64 | 0.240 |
| 10 cmH2O | 11.1 V | 0.598 | 10.01 | 53.88 | 0.268 |
| 10 cmH2O | 11.1 V | 0.764 | 10.04 | 65.33 | 0.303 |
| 10 cmH2O | 11.1 V | 0.213 | 10.04 | 0.00 | blocked |
| 12 cmH2O | 11.1 V | 0.410 | 12.03 | 30.33 | 0.179 |
| 12 cmH2O | 11.1 V | 0.470 | 11.99 | 37.36 | 0.200 |
| 12 cmH2O | 11.1 V | 0.619 | 12.02 | 50.75 | 0.240 |
| 12 cmH2O | 11.1 V | 0.727 | 12.00 | 59.11 | 0.268 |
| 12 cmH2O | 11.1 V | 0.930 | 12.03 | 71.22 | 0.303 |
| 12 cmH2O | 11.1 V | 0.243 | 12.03 | 0.00 | blocked |

### Example 3

This following table compares the radiated noise and delivered air flow attributes of two embodiments of the invention versus a commercially available ResMed Air Mini bedside CPAP device. Noise was measured at 1.0 m from blower unit exhausting through 0.18 inch plate orifice at end of 6 foot hose facing away from a sound meter microphone. Ambient Conditions—89° F., 1420 feet elevation, 20-22 dBA sound. The A2 unit is a system of the present invention powered by an external power supply and the B2 unit is similar system powered by a 3 cell lithium ion battery integrated into the device housing. The flow and noise characteristics are very similar when tested under identical conditions.

| Measurement | ResMed Air Mini | | A2 Unit | | B2 unit | |
|---|---|---|---|---|---|---|
| Pressure | * 9.3 | cmH2O | 10.0 | cmH2O | 10.0 | cmH2O |
| Flow | 26.5 | LPM | 27.8 | LPM | 27.6 | LPM |
| Noise | 32 | dBA | 32 | dBA | 31.5 | dBA |

* Air Mini set to 10 cmH2O pressure for use with its included small orifice mask

### Example 4

To gather real-world data on the acceptance of the PAP system of the invention by CPAP users, test subjects were asked to wear the PAP system and answer a series of questions. The following reports those results.
Demographics: 45 y/o male
Height, weight: 6′0″ 250 lbs
Years of experience with CPAP: 16 yrs
CPAP unit description: Unit without battery just external lead.
Pressure setting: 10 cm H₂O
Hours worn: 1 hour in office and 5-6 hours per day for 2 days
Perceptions of:
   Noise: Inconsequential (very low)
   Fit: Comfortable
   Stability when moving from back to side sleeping positions and moving head up and down—did device need repositioning: No, device did not need adjusting Comfort on chin and nose: Very comfortable
   Leaks: None
   Clearance from chest: Adequate
   Freedom from 6 foot CPAP hose: Yes
   Would you remove device at night to go to bathroom?: No
   Other comments: Unit is extraordinary with no need for hose and is portable; would be a great unit for travel as well as for everyday use.
Demographics: 82 y/o male
Height, weight: 5′6″ 199 lbs
Years of experience with CPAP: 2 yrs
CPAP unit description: Unit without battery just external lead.
Pressure setting: 6 cm H₂O
Hours worn: 0.6 hour in office
Perceptions of:
   Noise: Very good
   Fit: Comfortable except nose piece needed to be larger
   Stability when moving from back to side sleeping positions and moving head up and down—did device need repositioning: Very stable, device did not move Comfort on chin and nose: Good on chin, nose piece too small
   Leaks: None
   Clearance from chest: Good
   Freedom from 6 foot CPAP hose: Liked
   Would you remove device at night to go to bathroom?: No (and demonstrated by wearing to bathroom)
   Other comments: It's very comfortable and easy to move around with.

In the present tests, ambient sound was approximately 20 dB(A). The present devices comprise a compact air inlet path from outside the device to the inlet hole of the blower that contains a set of defined resonant chambers and flow straightening elements that provide a radiated blower sound pressure of about 32 dB(A). Under similar measurement conditions, the blower isolated from the acoustic dampening provided by the device elements was 44 dB(A).

All dimensions recited in the present application are exemplary only, and may be modified in accordance with the teachings of the invention. The terms “about” or “approximately” as used herein with regard to any value is +/−1% to 10% of the value. In preferred embodiments this may be +/−10% of the value, +/−5% of the value, or +/−2% of the value.

The present invention and the various features and advantageous details thereof are explained more fully with reference to the non-limiting embodiments that are illustrated in the accompanying drawings and detailed in the following description. It should be noted that the features illustrated in the drawings are not necessarily drawn to scale. Descriptions of well-known components and processing techniques are omitted so as to not unnecessarily obscure the present invention. The examples used herein are intended merely to facilitate an understanding of ways in which the invention may be practiced and to further enable those of skill in the art to practice the invention. Accordingly, the examples should not be construed as limiting the scope of the invention. In the drawings, like reference numerals designate corresponding parts throughout the several views.

Those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims:

1. A flow generator adapted for use in a PAP system, comprising:
a housing;
an air inlet aperture formed in a wall of the housing configured to allow atmospheric air into the housing;
an air path within the housing in fluid communication with the air inlet aperture, comprising
a first chamber proximal to the air inlet aperture having a substantially rectangular cuboid shape,
a plurality of flow straighteners, each flow straightener comprising an array of substantially parallel air passages, wherein the first flow straightener in the plurality is downstream of, and in fluid communication with, the first chamber, wherein the remaining flow straightener in the plurality are in serial fluid communication with the first flow straightener, and wherein a chamber follows each flow straightener in the plurality, wherein the chamber is an unoccupied space in the air path, and
a blower chamber within the housing immediately downstream of, and in fluid communication with, the chamber following the last flow straightener in the plurality;
a blower within the blower chamber comprising an impeller and a motor; and
an air outlet aperture formed in a wall of the housing operably connected to the blower to receive air exiting an air output portion of the blower,
wherein the blower, when energized, is configured to draw atmospheric air into the inlet aperture and thereby generate an airflow through the air path and into the blower, and direct the airflow at greater than atmospheric pressure to the air outlet aperture.

2. A flow generator according to claim 1, comprising:
a housing;
an air inlet aperture formed in a wall of the housing configured to allow atmospheric air into the housing;
an air path within the housing in fluid communication with the air inlet aperture, comprising
a first chamber proximal to the air inlet aperture having a substantially rectangular cuboid shape,
a first flow straightener comprising a first array of substantially parallel air passages downstream of, and in fluid communication with, the first chamber,
a second chamber immediately downstream of, and in fluid communication with, the first air straightener having a substantially rectangular cuboid shape, and
a second flow straightener comprising a second array of parallel air passages downstream of, and in fluid communication with, the second chamber,
a third chamber immediately downstream of, and in fluid communication with, the second air straightener, and
a fourth chamber within the housing downstream of, and in fluid communication with, the third chamber;
a blower within the fourth chamber comprising an impeller and a motor; and
an air outlet aperture formed in a wall of the housing operably connected to the blower to receive air exiting an air output portion of the blower,
wherein the blower, when energized, is configured to draw atmospheric air into the inlet aperture and thereby generate an airflow through the air path and into the fourth chamber, and direct the airflow at greater than atmospheric pressure to the air outlet aperture.

3. A flow generator according to claim 1, comprising:
a housing;
an air inlet aperture formed in a wall of the housing configured to allow atmospheric air into the housing;
an air path within the housing in fluid communication with the air inlet aperture, comprising
a first chamber proximal to the air inlet aperture having a substantially rectangular cuboid shape,
a first, second, and third flow straightener comprising a first array of substantially parallel air passages, wherein the first flow straightener is downstream of, and in fluid communication with, the first chamber, the second flow straightener comprising a second array of parallel air passages is downstream of, and in fluid communication with the first flow straightener, and the third flow straightener comprising a third array of parallel air passages is downstream of, and in fluid communication with the second flow straightener,
a chamber following each flow chamber, wherein the chamber is an unoccupied space in the air path, and
a blower chamber within the housing immediately downstream of, and in fluid communication with, the chamber following the third flow straightener;
a blower within the blower chamber comprising an impeller and a motor; and
an air outlet aperture formed in a wall of the housing operably connected to the blower to receive air exiting an air output portion of the blower,
wherein
the blower, when energized, is configured to draw atmospheric air into the inlet aperture and thereby generate an airflow through the air path and into the blower, and direct the airflow at greater than atmospheric pressure to the air outlet aperture.

4. A flow generator according to claim 3, wherein the spacing between the first and second flow straighteners and the spacing between the second and third flow straighteners are each independently between 0.1 and 0.3 mm, and
the first, second, and third flow straighteners together occupy between 75% and 99% of the volume between the air inlet aperture and the blower chamber, measured without accounting for the volume occupied by the array of parallel air passages in each flow straightener.

5. A flow generator according to claim 3, wherein the first flow straightener comprises seven air passages, the second flow straightener comprises six air passages, and the third flow straightener comprises seven air passages, resulting in a 7-6-7 pattern of preferably hexagonal air passages in the three flow straighteners, wherein one or more of the air passages in each flow straightener are substantially “in line” with corresponding air passages in an adjoining flow straightener, and at least one air passage in the “7” flow straightener is not in line with a corresponding air passage in the adjoining “6” flow straightener.

6. A flow generator according to claim 3, wherein each air passage in the plurality of flow straighteners is substantially identical in cross-section.

7. A flow generator according to claim 3, wherein each air passage in the plurality of flow straighteners is substantially hexagonal in cross-section.

8. A flow generator according to claim 2, having at least one of the following characteristics:
i) the volume of the first chamber is between 0.1 mm³ to 6,500 mm³,
ii) the volume of the second chamber is approximately 3,000 to 10,000 mm³,
iii) the volume of the third chamber ahead of an optional third air straightener is approximately 3,000 to 10,000 mm³,
iv) the volume of the final chamber connecting to the chamber containing the blower is approximately 4,000 to 14,000 mm³,
v) the length of each array of air passages in the air straightener elements is approximately 19 to 25 mm, and
vi) the cross-sectional area of each hexagonal flow passage in the first, second and optional third array of air passages is approximately 8 mm².

9. A flow generator according to claim 8 having each of characteristics i) through vi).

10. A flow generator according to claim 8, wherein one or more air passages in each of flow straightener in the plurality is in line with a corresponding air passage with the other flow straighteners in the plurality.

11. A flow generator according to claim 10, wherein the central axis of the air path through the plurality of flow straighteners is approximately linear, and the central axis of the air path from the chamber following the final flow straightener in the plurality to the blower chamber diverges from the central axis of the air path from the first flow chamber to the third flow chamber.

12. (canceled)

13. (canceled)

14. A flow generator according to claim 11, wherein the blower chamber comprises a resilient liner, wherein the blower is adapted to seat in a raised retaining feature integrally or unitarily formed on a surface of the resilient liner.

15. A PAP system adapted for treatment of an individual having respiratory disease, sleep disordered breathing, or requiring breathing support, comprising:
a flow generator, comprising
a housing,
a blower comprising an impeller and a motor within the housing, and
a flow outlet within the housing, wherein the flow generator is adapted to provide a flow of pressurised breathable gas produced by the blower through the flow outlet;
a power supply operably connected to the flow generator and adapted to provide electrical energy to energize the blower;
a securement interface, comprising
a bracket projecting from the flow generator or the power supply, wherein the bracket is adapted to support and orient the flow generator or the power supply on the mental protuberance of the individual's mandible and to isolate the individual's cranial bones from noise and vibration generated during operation of the flow generator,
at least two slots on the bracket configured and arranged to be on opposite sides of individual's the mental protuberance,
a one or more flexible head straps adapted to couple to the at least two slots on the bracket and to engage the head of the individual to secure the bracket on the individual's the mental protuberance;
a patient interface, comprising
an interface portion in fluid communication with the flow outlet of the flow generator, and
a face mask adapted to administer airflow produced by the flow generator into an airway of the individual.

16-19. (canceled)

20. A PAP system according to claim 15, wherein the face mask comprises a pair of nasal pillows adapted for at least partial insertion into the nares of the individual.

21. A PAP system according to claim 15, wherein the bracket projecting from the flow generator comprises a chin cup adapted to conform approximately to the shape of the mental protuberance, and a chin cup insert which is adapted to form a cushioned contact surface between the chin cup and the mental protuberance.

22-26. (canceled)

27. A face mask adapted for use with a PAP system, comprising:
a hollow body comprising an inner chamber and formed of a resilient material forming a c-shaped cavity configured to receive a patient's nose and to form a seal against the patient's nose;
a flow generator interface portion configured for fluid communication between an air flow outlet of the PAP system and the inner chamber of the hollow body; and
one or more air outlet openings through the hollow body within the c-shaped cavity configured to direct air flow entering the hollow body from the PAP system towards the nares of the patient.

28. (canceled)

29. A method of treating sleep apnea, snoring, or another breathing disorder in an individual, comprising:
securing a PAP system of claim 15 to the individual by positioning the bracket projecting from the flow generator onto the mental protuberance of the individual's mandible, engaging the head straps to the head of the individual to maintain the flow generator on the mental protuberance, and positioning the face mask over an external airway passage of the individual; and
energizing the flow generator to administer airflow produced by the flow generator into the individual's airway via the face mask.

30. A PAP system adapted for treatment of an individual having respiratory disease, sleep disordered breathing, or requiring breathing support comprising a flow generator configured to engage onto the mental protuberance of the individual's mandible and be retained thereon by head straps.

31. A PAP interface comprising:
a bulbous, hollow chamber comprising an air inlet hole configured to operably connect to a PAP system to receive airflow produced thereby, a pair of leaflets which wrap around the exterior of an individual's nose and thereby position at least one air exit hole in the hollow chamber at each nares of the individual, and one or more vent holes, wherein the bulbous, hollow chamber is formed from a 40 to 70 durometer biocompatible material configured to provide a compressible compliance buffer between the nose and the PAP system.
